(19) United States
(12) Patent Application Publication
(10) Pub. No.: US 2022/0025428 A1
(43) Pub. Date: Jan. 27, 2022

(54) NUCLEIC ACID MEMORY (NAM) / DIGITAL NUCLEIC ACID MEMORY (DNAM)

(71) Applicant: Boise State University, Boise, ID (US)

(72) Inventors: William Hughes, Boise, ID (US); Elton Graugnard, Boise, ID (US); Wan Kuang, Meridian, ID (US); Timothy Andersen, Boise, ID (US); Eric Hayden, Boise, ID (US); Reza Zadegan, Greensboro, NC (US); George Dickinson, Boise, ID (US); Luca Piantanida, Boise, ID (US); William Clay, Boise, ID (US); Christopher Green, Alexandria, VA (US); Golam Mortuza, Boise, ID (US)

(21) Appl. No.: 17/443,312

(22) Filed: Jul. 23, 2021

Related U.S. Application Data
(60) Provisional application No. 62/705,995, filed on Jul. 24, 2020.

(51) Int. Cl. C12Q 1/68; G06N 3/12

(52) U.S. Cl. CPC C12Q 1/68; G06N 3/123

(57) ABSTRACT

Compositions and methods for encoding and retrieving data into nucleic acid memory for storage. More specifically, data is encoded into spatial locations within a nucleic acid architecture, which allows the data to be retrieved using super resolution microscopy. The data is then interrogated for errors, the errors corrected, and the data is then decoded.

### CROSS REFERENCE TO RELATED APPLICATION

The present application claims priority to the earlier filed U.S. Provisional Application having Ser. No. 62/705,995, and hereby incorporates subject matter of the provisional application in its entirety.

### GRANT REFERENCE

This invention was made with government support under Grant No. 1807809, awarded by the National Science Foundation. The Government has certain rights in the invention.

### FILED OF THE INVENTION

The invention relates generally to nucleic acid memory (NAM). More specifically, the invention relates to digital Nucleic Acid Memory (dNAM) which use a nucleic acid architecture to create a physical address by providing docking sites for single stranded nucleic acid for information processing. The invention further relates to methods for enhanced data retention and retrieval and systems for use.

### BACKGROUND OF THE INVENTION

Archival memory materials are quickly approaching their physical and economic limits. Currently, the most widely used material for this purpose is magnetic tape. Recent advancements in magnetic tape report a two-dimensional areal information density up to 31 Gbit/cm², though the current commercially available material typically has lower density. New non-volatile memory materials are needed due to the rapid growth of the global datasphere and environmental impacts. DNA may be a viable option to magnetic tape because of its potential for vast information density, significant retention time, and low energy of operation. As a sustainable alternative, in terms of durability, typical magnetic tape lasts for 10-30 years, while double stranded DNA is estimated to be stable for millions of years under optimal environmental conditions.

Due to advances in synthesizing and sequencing DNA, the cost related to high throughput sequences has greatly dropped. As synthesis and sequencing of DNA becomes cheaper, this has focused the use of DNA as information storage on storing the data within the sequence and relying upon sequences to extract the data later. However, other options may be available.

DNA nanotechnology has been used to create a variety of one-, two-, and three-dimensional architectures resulting in unprecedented control of both the placement and spacing of nanoparticles, such as dyes, quantum dots, and gold nanoparticles. For example, gold nanoparticles may be attached to DNA bricks or DNA staples or other architectures to place them into lines or other shapes after the architectures self-assemble. However, imaging the nanostructures with sufficient detail to possibly distinguish the individual nanoparticles was not possible until the recent advancements in super-resolution microscopy.

Accordingly, it is an aspect of the present disclosure to disclose the use of nucleic acid architectures coupled with a dye to be used for nucleic acid memory (NAM). Another aspect of the present disclosure is to further digitize the stored information into digital nucleic acid memory (dNAM). In a further aspect of the present disclosure is to retrieve the data encoded on a nucleic acid architecture and check and correct it for errors prior to decoding the stored information.

These and other objects, advantages and features of the present disclosure will become apparent from the following specification taken in conjunction with the claims set forth herein.

### BRIEF SUMMARY OF THE INVENTION

Applicants have created compositions of nucleic acid architectures that may act as optical breadboards with data sites having nanometer spacing. The breadboards self-assemble and may use any type of nucleic acid architectures, such as but not limited to nucleic acid origami or molecular canvas. In an aspect, the staple strands or bricks are arranged at addressable locations that define an indexed array of digital information. These staple strands or bricks are also referred to as data strands. Reading this site-specific localization of digital information is enabled by designing data strands with nucleotides that extend from the architecture. Extended staple strands have two domains: the first domain forms a sequence-specific double helix with the architecture and determines the address of the data; the second domain, which is optional, extends above the architecture and, if present, provides a docking site for a labelled single-stranded DNA imager strand. Binary states of the data sites are defined by the presence (1) or absence (0) of the data domain, which is read with microscopy, such as super resolution (SRM). In another aspect, unique patterns of binary data are encoded by selecting which staple strands have and do not have data domains. As an integrated memory platform, data is entered into dNAM when the data strands encoding 1 or 0 are selected for each addressable site. The data strands are then stored directly, or self-assembled and stored. Editing data is achieved by replacing specific strands or the entire content of a stored structure. To read the data, the origami may be optically imaged below the diffraction limit of light.

In another aspect, error-correcting algorithms are used to ensure error-free data recovery. Detection of individual nucleotide molecules using SRM is routinely limited by incomplete staple strand incorporation, defective imager strands, fluorophore bleaching, and background fluorescence. In one embodiment, the signal-to-noise ratio is improved by averaging multiple images of identical structures. In a more preferred embodiment, encoding and decoding algorithms that combine fountain codes with bi-level, parity-based, and orientation-invariant error detection scheme may be utilized. Fountain codes enable transmission of data over noisy channels. They work by dividing a data file into smaller units called droplets and then sending the droplets at random to a receiver. Droplets can be read in any order and still be decoded to recover the original file, so long as a sufficient number of droplets are sent to ensure that the entire file is received. In an embodiment, each droplet is encoded onto a single origami and additional bits of information are added for error correction to ensure that individual droplets will be recovered, in the presence of high noise, from individual origami. Together, the error correction and fountain codes increase the probability that the message is fully recovered while minimizing the number of nucleotide origami that must be observed. In other embodiments, machine learning algorithms, such as but not limited to, supervised learning, unsupervised learning, or reinforcement learning algorithms may be used for any step or every step of the error correction, encoding, and/or decoding the NAM or dNAM.

The forgoing summary is illustrative only and is not intended to be in any way limiting. In addition to the illustrative aspects, embodiments and features described above, further aspects, embodiments, and features of the present technology will become apparent to those skilled in the art from the following drawings and the detailed description, which shows and describes illustrative embodiments of the present technology. Accordingly, the figures and detailed description are also to be regarded as illustrative in nature and not in any way limiting.

### BRIEF DESCRIPTION OF THE DRAWINGS

FIG. 1A is an illustration representation of a binary dNAM nucleotide nanostructure or architecture, a nucleotide nanostructure with specific sequences used to localize data strands (a.k.a. information-bearing particles) to programmable sites within the DNA nucleotide nanostructure. Site-specific localization is enabled by extending/not-extending the structural staple strands of the nucleotide nanostructure to create physical representations of 1s/0s. The presence, absence, and identity of a data strand's docking sequence defines the state of each data strand and is assessed by monitoring the binding of data imager strands via super-resolution microscopy (SRM). FIG. 1B is an illustration representation of a one of the 15 designs used to enable reading of the test message, “Data is in our DNA!\n”. The colors of the matrix sites depicted in the design correspond with the roles of the site's bit values as follows: droplet (green), parity (blue), checksum (yellow), index (red), and orientation (magenta). FIG. 1C is an overview of the how to ‘read’ the message, 4 μL of the DNA-nucleotide nanostructure mixture, containing 0.33 nM of each nucleotide nanostructure, was imaged using DNA-PAINT. The nucleotide nanostructure in the rendered image were identified and converted to an array of 1's and 0's corresponding to the pattern of localizations seen at each matrix location. The decoding algorithm performed error correction where possible, and successfully retrieved the entire message when sufficient data droplets and indexes were recovered. Scale bar, 100 nm.

FIG. 2A is an exemplary flowchart representing an example of designing the nucleotide nanostructure or architecture, showing the initial steps for encoding process: converting the text to binary data string, splitting the string into segments; combining the segments using XOR operator into droplets; assigning an index to each droplet, and adding orientation markers. FIG. 2B is a flowchart representing an example of calculating the checksum bits from symmetrically positioned matrix edge values. FIG. 2C is a flowchart representing an example FIG. 2C is a flowchart representing an example of calculating the parity bits from symmetrically positioned edge values and checksum. FIG. 2D is a representation of synthesizing and assembling the nucleotide nanostructure based on the design calculated in FIGS. 2A to 2C and its storage. FIG. 2E is a pictorial representation of reading the stored nucleotide nanostructure. FIGS. 2A-2E show the twelve steps involved in encoding a text message using dNAM. The encoding process depicts the proof-of-principle experiment described herein, showing the design process for one of the 15 origami, as an example.

FIG. 3A is a representative design of SRM imaging, DNA-PAINT, of digital nucleic acid memory (dNAM) indicates all sites are recovered in a single read dNAM nucleotide nanostructure from a DNA-PAINT recording were identified and classified by aligning and template matching them with the 15 design matrices in which all potential docking sites are shown, filled circles indicate sites encoded ‘0’ (dark grey) or ‘1’ (white). Colored boxes indicate the regions of the matrices used for the droplet (green), parity (blue), checksum (yellow), index (red), and orientation (magenta). For clarity, only the first design image includes the colored matrix sites. FIG. 3B is a pictorial representation of the ‘Averaged’ images of 4560 randomly selected nucleotide nanostructure, grouped by index, are depicted right (DNA-PAINT). Scale bar, 10 nm.

FIG. 4A is a pictorial and graphical representation of the full width half maximum (FWHM) values of the transect measurements centered on binding sites in rendered images of individual nucleotide nanostructures. FIG. 4B is the FWHM for ‘averaged’ dNAM nucleotide nanostructures. FIGS. 4A and 4B show the transects placed horizontally (as shown in red) on vertically for measurements. A plot from a single binding site is shown with a Gaussian fit to the data plotted in red. FIG. 4C is a graphical representation of the Gaussian fits for binding sites from each experiment are plotted in grey for single structures. FIG. 4D is a graphical representation of ‘averaged’ structures (after centering and normalization). The mean of the grey lines is shown in black. The inset plots are the representative results from a single experimental recording. The mean FWHM value for individual fits to each experiment was calculated and reported in the main text. Origami-6 was used in all cases, as it was the most consistently recovered structure. Scale bars, 10 nm.

FIG. 5 is a representation of atomic force microscopy (AFM) images of all 15 dNAM “Data is in our DNA!/n” nucleotide nanostructure showing docking sites. An inverse FFT analysis with a band rejected filter has been applied to highlight the docking positions in right-hand panels. Every image is 90×110 nm² and the color scale ranges over 250 pm.

FIG. 6 is an exemplary flowchart demonstrating the operations for message recovery showing the main steps involved in decoding a message from dNAM. First, each individual origami captured in a DNA-PAINT recording is converted into a binary string (Image Processing). Next, errors in each binary string are detected and corrected if possible (Error Correction) using the algorithm described in Flowchart 1 (FIG. 7) and index and droplet data extracted. Finally, segment information is retrieved from the droplets (Segment Information Extracted) pooled with data from other origami and passed to the fountain code decoding algorithm shown in Flowchart 2 (FIG. 7), which reassembles the original file (Fountain Code Decoding).

FIG. 7 is an exemplary flowchart demonstrating the operations for error correction. A flowchart depicting the operations performed by the error correction algorithm for an individual origami is shown. A priority queue is initialized with an individual origami m (the working_matrix). Based on the parity and checksum bits mismatch, the algorithm deduces a set of probable errors and a matrix weight for the working matrix. The matrix weight is proportional to the number of errors, and the main goal of the algorithm is to reduce the matrix weight in a greedy fashion. To that end, each of the probable errors in the working_matrix is sequentially flipped, and a matrix weight calculated for every resulting matrix. The two resulting matrices with the lowest weights are enqueued. The algorithm then replaces the working_matrix with the recalculated matrix possessing the lowest matrix weight from the queue. If the current working matrix already has 9 bits flipped it is discarded and the next matrix in the queue used. The algorithm repeats these steps until the matrix weight equals zero, at this point the data in the origami is considered to have been error-corrected and is passed to the next stage of the decoding (Accept). If the priority queue is emptied before the matrix weight reaches zero, the origami data is considered unrecoverable and is removed from the analysis (Reject).

FIG. 8A is a representation of the array positions of nucleotide nanostructure (only considering structures with 15 or less errors, as identified by template matching) were classified as either ‘outer’, ‘mid’ or ‘inner’ depending on their position in the array. FIG. 8B is a graphical representation of the normalized false negative mean error for each classification was calculated and normalized by dividing by the overall mean error for that zone. FIG. 8C is a graphical representation of the normalized false positive mean error for each classification was calculated and normalized by dividing by the overall mean error for that zone. Mean values for three experiments are shown. Error bars indicate ±SD.

FIG. 9A is a graphical representation of each index observed in a single recording, based on template matching. The mean counts are shown as black bars, percentage of total dNAM nucleotide nanostructure are shown in red. FIG. 9B is the mean number of total errors, grouped into false negatives, and grouped into false positive for each structure. FIG. 9C is a graphical representation of the percent of nucleotide nanostructure passed to the decode algorithm that had both their indexes and data strings correctly identified. FIG. 9D is a graphical representation of the percentage of each nucleotide nanostructure decoded plotted against the mean number of errors for each structure. FIG. 9E is a histograms representation of the total mean numbers of errors found in nucleotide nanostructure identified by template matching (open bars) and the decode algorithm (black bars) are shown. The difference between the two is plotted in blue. Mean values for three experiments are depicted in all graphs, error bars indicate ±5D.

FIG. 10 is a graphical representation of the mean number of unique dNAM nucleotide nanostructure matrices correctly decoded for randomly selected subsamples of decoded binary strings. The analysis was further broken out by the number of errors corrected for each nucleotide nanostructure, three examples are plotted (1, 5 and 9). Black filled circles depict the results for nine error corrections, which is the ‘maximum allowable number of errors’ parameter used in the decode algorithm for all other analysis reported here. The horizontal lines indicate the probability of recovering the message with different numbers of unique droplets. With fourteen or more droplets, the message should always be recovered (thick green line, and above indicates 100% chance of recovery) and with nine or fewer droplets the message will never be recovered (thick red line and below indicates 0% chance of recovery). Mean values for three experiments are shown. Error bars indicate ±SD.

FIG. 11A is a graphical representation of the mean number of dNAM nucleotide nanostructure needed to successfully recover messages of increasing length with (open circles) or without (filled squares) redundant bits based on simulations to determine the theoretical success rates for correctly decoding individual dNAM nucleotide nanostructure and recovering encoded messages. FIG. 11B is a graphical representation of the mean success for recovering both individual nucleotide nanostructure (circles) and the entire message (squares) are plotted against the mean number of errors per nucleotide nanostructure (randomly generated for simulated data). Simulation recovery rates (filled symbols) are averages of all message sizes tested (160 to 12,800 bits). For experimental data (open circles) the mean success was estimated by comparing the decode algorithm's results with that of the template-matching algorithm. Two types of dNAM nucleotide nanostructure were simulated, with (open circles) and without (black squares) redundancy.

FIG. 12 is an exemplary flowchart demonstrating the operations performed to recover file segment data from droplet data. A flowchart demonstrating the operations performed by the fountain decoding algorithm to recover file segment data from droplet data is shown. After retrieving the binary data from the dNAM origami images, the dNAM decoding algorithm corrects errors in the binary data and extracts both the index and droplet information. The droplets are collected (Droplet Table), with each droplet containing one or more file segments. The data in single degree droplets, such as D9 and D8, encode single segments and are used directly to reconstruct the file (Recovered File). To extract additional individual segment data from multi-segment droplets, the decoding algorithm performs a series of XOR operations. The index information allows the algorithm to determine both the degree of the droplet and which segments of the file that the droplet encodes. Taking the case of D2, a series of XOR operations must be performed in order to retrieve additional segment data from it. The decoding algorithm may XOR a multi-degree droplet with another droplet if the other droplet's segment(s) are a proper subset of the multi-degree droplet. For example, the segments contained in D6 are a proper subset of those in D2. After XORing D2 and D6 a new droplet is generated containing segments S5 and S6, which ultimately leads to the algorithm extracting the data for S6. This process is repeated in a greedy fashion until the algorithm retrieves all of the file's segment data (Recovered File), or it runs out of options for XORing droplets (in which case the entire file cannot be successfully recovered). For simplicity, only six of the 15 possible droplets are shown, with the resulting recovered segments depicted in colored boxes (Recovered Segments).

### DETAILED DESCRIPTION

Unless otherwise defined herein, scientific and technical terms used in connection with the invention shall have the meanings that are commonly understood by those of ordinary skill in the art. Further, unless otherwise required by context, singular terms shall include the plural and plural terms shall include the singular. Generally, nomenclatures used in connection with, and techniques of, biochemistry, enzymology, molecular and cellular biology, microbiology, genetics and protein and nucleic acid chemistry and hybridization described herein are those well-known and commonly used in the art. The methods and techniques are generally performed according to conventional methods well known in the art and as described in various general and more specific references that are cited and discussed throughout the present specification unless otherwise indicated.

### Definitions

The following terms, unless otherwise indicated, shall be understood to have the following meanings:

It should be noted that, as used in this specification and the appended claims, the singular forms “a,” “an,” “said,” “another,” and “the” include plural referents unless the content clearly dictates otherwise. Thus, for example, reference to a composition containing “a compound” includes a mixture of two or more compounds. It should also be noted that the term “or” is generally employed in its sense including “and/or” unless the content clearly dictates otherwise.

Numeric ranges recited within the specification are inclusive of the numbers defining the range and include each integer within the defined range. Throughout this disclosure, various aspects of this invention are presented in a range format. It should be understood that the description in range format is merely for convenience and brevity and should not be construed as an inflexible limitation on the scope of the invention. Accordingly, the description of a range should be considered to have specifically disclosed all the possible sub-ranges, fractions, and individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6, and decimals and fractions, for example, 1.2, 3.8, 1½, and 4%. This applies regardless of the breadth of the range.

Other than in the operating examples, or where otherwise indicated, all numbers expressing quantities of ingredients or reaction conditions used herein are to be understood as being modified in all instances by the term “about”.

As used herein, the term “about” modifying the quantity of an ingredient in the compositions of the invention or employed in the methods of the invention refers to variation in the numerical quantity that can occur, for example, through typical measuring and liquid handling procedures used for making concentrates or use solutions in the real world; through inadvertent error in these procedures; through differences in the manufacture, source, or purity of the ingredients employed to make the compositions or carry out the methods; and the like. The term about also encompasses amounts that differ due to different equilibrium conditions for a composition resulting from a particular initial mixture. Whether or not modified by the term “about,” the claims include equivalents to the quantities.

“Non-covalent” refers to any molecular interactions that are not covalent—i.e. the interaction does not involve the sharing of electrons. The term includes, for example, electrostatic, π-effects, van der Waals forces, and hydrophobic effects. “Covalent” refers to interactions involving the sharing of one or more electrons.

As used herein, a “structural strand” is a strand of nucleic acid comprised of any synthetic or natural nucleotide that may be of any shape or size used to provide structure to a nucleic acid architecture. By way of non-limiting example, bricks, staples, and scaffolds are structural strands in a nucleic acid architecture.

As used herein, a “brick” or a “nucleotide brick” is a structural strand. The terms “brick” and “nucleotide brick” are used interchangeably herein.

As used herein, a “nucleotide” is any nucleoside linked to a phosphate group. The nucleoside may be natural, including but not limited to, any of cytidine, uridine, adenosine, guanosine, thymidine, inosine (hypoxanthine), or uric acid; or synthetic, including but not limited to methyl-substituted phenol analogs, hydrophobic base analogs, purine/pyrimidine mimics, icoC, isoG, thymidine analogs, fluorescent base analogs, or X or Y synthetic bases. Alternatively, a nucleotide may be abasic, such as but not limited to 3-hydroxy-2-hydroxymethyl-tetrahydrofuran, which act as a linker group lacking a base or be a nucleotide analog.

As used herein, “nucleotide duplex” is when two strands of nucleotide oligomers complementary bind to each other. The two strands may be part of the same nucleotide molecule or separate nucleotide molecules. Complementation may either be total binding of an entire strand or partial, such as a specific section of a strand binding to different section. The second section may be on the same or different strand.

As used herein, “nucleotide origami” or “origami” is two or more structural strands, where one brick is a “scaffold” and provides the main body of the overall structure and is bound by one or more “staple(s).”

As used herein, a “scaffold” is a single stranded structural strand which may be rationally designed to self-assemble into hairpin loops, helical domains, and locking domains. The scaffold may use staples to direct the folding and to hold the final shape. Alternatively, the scaffold may use intrinsic self-complementary pairing to hold the final shape.

As used herein, a “staple” or “staple strand” is a structural strand which pairs with a longer main body brick in nucleotide origami to help fold the main body brick into the desired shape.

As used herein, a “nanobreadboard,” “breadboard,” “substrate,” or “template” is a total or final structure of a nucleic acid structure or shape. For example, a mobile or immobile 4-arm junction, origami happy face, rectangular brick, or double stranded DNA (dsDNA) in its final structure.

As used herein, an “architecture” is a one-, two-, or three-dimensional structure built using one or more structural strands. As used herein, a “nucleic acid architecture” is a one-, two-, or three-dimensional structure built using one or more structural strands. Examples include nucleotide origami or molecular canvases. As used herein, “nucleic acid architecture” and “nucleic acid nanostructure” are used interchangeably. As used herein, “architecture” and “nanostructure” are used interchangeably.

As used herein, “self-assembly” refers to the ability of nucleotides to adhere to each other, in a sequence-specific manner, in a predicted manner and without external control.

As used herein, Førster resonance energy transfer (FRET), fluorescence resonance energy transfer (FRET), resonance energy transfer (RET), or electronic energy transfer (EET) refers to energy transfer between two light-sensitive molecules (donor and acceptor chromophores) or aggregates thereof.

As used herein, the term “dye” refers to a molecule comprising a “chromophore” or a “fluorophore.” As the chromophore or fluorophore may comprise the entire molecule, “dye”, “chromophore”, and “fluorophore” may be used interchangeably with each other unless otherwise specified.

As used herein, “indexed array” refers to a nucleic acid architecture comprising structural strands, such as a staple strands or data strands, which may or may not extend out from the nucleic acid architecture and are designed to localize to readable positions, an “indexed position”, along the nucleic acid architecture.

As used herein, “archival storage,” “long-term storage,” and “stable storage” refers to the storage of inactive data. Typically, inactive data is data that may be rarely accessed or may need to be retained for long periods of time.

As used herein, “binary string” refers to a sequence of bits (i.e., a sequence of 0's and 1's). It can also be used to describe a sequence of bytes—for example, for an 8-bit byte a sequence in which every element is 8-bits long.

As used herein, a “bit” refers to a binary digit, the smallest unit of information used by a computer. In dNAM, a bit is encoded by the data strand.

As used herein, a “byte” refers to the smallest addressable unit of memory used by a computer, made up of bits (typically 8) and originally used to encode a single character of text.

As used herein, a “checksum bit” refers to a bit of the matrix which contains the checksum value from a subset of data bits, orientation bits, and indexing bits.

As used herein, a “data bit” refers to a bit of the matrix which contains a bit of information from segments of the message being encoded.

As used herein, a “data strand” or “information-bearing particles” refers to selected staple strands, bricks, or tiles within a NAM or dNAM architecture that are used to encode information. Data strands representing a zero (0) consist of only the staple strand, brick, or tile domain. Data strands representing a one (1) consist of the staple strand, brick, or tile domain extended by a docking domain, which acts as a docking site for complementary data imager strands. A single stranded oligomer may be modified to comprise of docking domains. Data strands are the information bearing particles in the architecture, analogous to the magnetic particles coating a tape or disk used in a tape recorder or hard drive for magnetic recording.

As used herein, a “docking site” or “docking domain” refers to segment of the data strand that is at least partially complement to the image strand to allow binding.

As used herein, a “decoding algorithm” refers to the algorithm used to decode messages from individual matrixes.

As used herein, “degree distribution” refers to the distribution of the segments into the droplet.

As used herein, “digital nucleic acid memory” (dNAM or digital NAM, used interchangeably herein) refers to a type of nucleic acid memory (NAM) in which information is encoded into defined spatial arrangements of DNA sequences on top of addressable DNA origami nanostructures.

As used herein, “dNAM origami” refers to a single rectangular 2D DNA origami nanostructure with specific sequences used to localize data strands to specific sites on the upper surface. This site-specific localization is enabled by extending (1) or not extending (0) the structural staple strands of the DNA origami to create addressable data strands. As used herein, “dNAM origami” and “dNAM nucleotide nanostructure” may be used interchangeably.

As used herein, “droplet” refers to a chunk of data created by a fountain code during transmission of a larger message.

As used herein, “greedy algorithm” refers to a type of algorithm that attempts to determine a globally-optimal solution to a problem by making locally-optimal choices at each search step. It uses a heuristic to determine each choice, such as: always choose the smallest, largest, etc.

As used herein, “imager strand” refers to a dye labelled, single strand of nucleic acid with a at least partially complementary docking domain corresponding at least one docking domain of a data strand that encodes a one (1) in a dNAM architecture. In dNAM, imager strands act as the read head and reveal the location of the ones in the dNAM architecture. To increase the thermo-mechanical stability, the imager strands may incorporate a hairpin loop. By increasing the thermo-mechanical stability, it is possible to probe shorter data strands.

As used herein, “structural strand” refers to a nucleic acid strand which is used to provide structure to the architecture when the architecture has self-assembled.

As used herein, “index bit” refers to a bit of the matrix that is used to encode a unique identifier for each droplet that allows the algorithm to determine the exact message segments that are encoded in the matrix.

As used herein, “matrix” refers to the 2-dimensional representation of the binary data, index, orientation marker, parity, and checksum bits encoded on the DNA.

As used herein, “Nucleic Acid Memory” (NAM) refers to a memory-storage material comprised of nucleic acids, or nucleotides, that has the potential for high volumetric density, long retention times, and low energy of operation.

As used herein, “orientation bit” refers to a bit of the matrix which indicates the orientation of the matrix.

As used herein, “packet” refers to a unit of data made into a single package for transmission over a digital network.

As used herein, “parity bit” refers to a bit of the matrix which contains the XORed value from a subset of data bits, orientation bits, indexing bits, and checksum bits, providing a second level of error correction capability.

As used herein, “matrix weight” refers to a float value calculated using the parity and checksum bits that indicates the presence of an error in the matrix.

As used herein, “priority queue” refers to a queue data type with each element in the queue has a priority value assigned. Abbreviated to pqueue here. Elements with high priority are served before elements with low priority.

As used herein, “read head” refers to the component of a recording device that senses the information stored in a memory material. Typically, an electromechanical mechanism that converts the magnetic field of a section of tape or disk platter into an electrical current. In dNAM the microscope or imager strands act as read heads.

As used herein, “composite bit” refers to a bit of data which is generated from the information presented at more than a given location within an architecture.

As used herein, “XOR operation” refers to the binary exclusive OR operation (⊕) in which corresponding bits of a binary number are compared and yields true (1) if exactly one of two conditions is true (false=0), see Table 1. For multiple arguments, XOR is defined to be true if an odd number of its arguments are true, and false otherwise (equivalent to addition modulo 2). See Table 2 for a three-argument function.

TABLE 1

| a | b | a ⊕ b |
|---|---|---|
| 0 | 0 | 0 |
| 0 | 1 | 1 |
| 1 | 0 | 1 |
| 1 | 1 | 0 |

TABLE 2

| a | b | c | a ⊕ b ⊕ c |
|---|---|---|---|
| 1 | 1 | 1 | 1 |
| 1 | 1 | 0 | 0 |
| 1 | 0 | 1 | 0 |
| 1 | 0 | 0 | 1 |
| 0 | 1 | 1 | 0 |
| 0 | 1 | 0 | 1 |
| 0 | 0 | 1 | 1 |
| 0 | 0 | 0 | 0 |

### Nucleic Acid Architecture

Nucleotide nanotechnology can be used to form complicated one-, two-, and three-dimensional architectures. The nucleotide nanostructures or architectures may comprise of one or more structural strands. The structural strands are designed to use the Watson-Crick pairing of the nucleotides to cause the bricks to self-assemble into the final and predictable architectures. Any method of designing the architectures and self-assembly may be used, such as but not limited to nucleotide origami, nucleotide brick molecular canvases, single stranded tile techniques, or any other method of nucleotide folding or nanoassembly such as, but not limited to, using nucleotide tiles, nucleotide scaffolds, nucleotide lattices, four-armed junction, double-crossover structures, nanotubes, static nucleotide structures, dynamically changeable nucleotide structures, or any other synthetic biology technique (as described in U.S. Pat. No. 9,073,962, U.S. Pub. No.: US 2017/0190573, U.S. Pub. No.: US 2015/0218204, U.S. Pub. No.: US 2018/0044372, or International Publication Number WO 2014/018675, each of which is incorporated in its entirety by reference).

The nucleobase making up the bricks may be natural, including but not limited to, any of cytosine, uracil, adenine, guanine, thymine, hypoxanthine, or uric acid; or synthetic, including but not limited to methyl-substituted phenol analogs, hydrophobic base analogs, purine/pyrimidine mimics, icoC, isoG, thymidine analogs, fluorescent base analogs, or X or Y synthetic bases, or other synthetic bases. Alternatively, a nucleotide may be abasic, such as but not limited to 3-hydroxy-2-hydroxymethyl-tetrahydrofuran, or alternatively a nucleotide analog may be used.

Non-limiting examples of synthetic nucleobases and analogs include, but are not limited to methyl-substituted phenyl analogs, such as but not limited to mono-, di-, tri-, or tatramethylated benzene analogs; hydrophobic base analogs, such as but not limited to 7-propynyl isocarbostyril nucleoside, isocarbostyril nucleoside, 3-methylnapthalene, azaindole, bromo phenyl derivates at positions 2, 3, and 4, cyano derivatives at positions 2, 3, and 4, and fluoro derivates at position 2 and 3; purine/pyrimidine mimics, such as but not limited to azole hetercyclic carboxamides, such as but not limited to (1H)-1,2,3-triazole-4-carboxamide, 1,2,4-triazole-3-carboxamide, 1,2,3-triazole-4-carboxamide, or 1,2-pyrazole-3-carboxamide, or heteroatom-containing purine mimics, such as furo or theinopyridiones, such as but not limited to furo[2,3-c]pyridin-7(6H)-one, thieno[2,3-c]pyridin-7(6H)-one, furo[2,3-c]pyridin-7-thiol, furo[3,2-c]pyridin-4(5H)-one, thieno[3,2-c]pyridin-4(5H)-one, or furo[3,2-c]pyridin-4-thiol, or other mimics, such as but not limited to 5-phenyl-indolyl, 5-nitro-indolyl, 5-fluoro, 5-amino, 4-methylbenzimidazole, 6H,8H-3,4-dihydropropyrimido[4,5-c][1,2]oxazin-7-one, or N⁶-methoxy-2,6-diaminopurine; isocytosine, isoquanosine; thymidine analogs, such as but not limited to 5-methylisocytosine, difluorotoluene, 3-toluene-1-β-D-deoxyriboside, 2,4-difluoro-5-toluene-1-β-D-deoxyriboside, 2,4-dichloro-5-toluene-1-β-D-deoxyriboside, 2,4-dibromo-5-toluene-1-β-D-deoxyriboside, 2,4-diiodo-5-toluene-1-β-D-deoxyriboside, 2-thiothymidine, 4-Se-thymidine; or fluorescent base analogs, such as but not limited to 2-aminopurine, 1,3-diaza-2-oxophenothiazine, 1,3-diaza-2-oxophenoxazine, pyrrolo-dC and derivatives, 3-MI, 6-MI, 6-MAP, or furan-modified bases.

Non-limiting examples of nucleotide analogs include, but are not limited to, phosporothioate nucleotides, 2′-O-methyl ribonucleotides, 2′-O-methoxy-ethyl ribonucleotides, peptide nucleotides (PNA), N3′-P5′ phosphoroamidate, 2′-fluoro-arabino nucleotides, locked nucleotides (LNA), unlocked nucleotides (UNA), bridge nucleotides (BNA), click nucleic acids (CNA), morpholino phosphoroamidate, cyclohexene nucleotides, tricyclo-deoxynucleotides, or triazole-linked nucleotides.

The nucleotides can then be polymerized into oligomers. The design of the oligomers will depend on the design of the final architecture. Simple architectures may be designed by any methods. However, more complex architectures may be design using software such as, but not limited to, caDNAno (as described at http://cadnano.org/docs.html, and herein incorporated by reference in its entirety), to minimize errors and time. The user may input the desired shape of the architecture into the software and once finalized, the software will provide the oligomer sequences of the bricks to create the desired architecture.

In some embodiments the architecture is comprised of nucleotide brick molecular canvases, wherein the canvases are made of 1 to 15,000 nucleotide bricks comprising of nucleotide oligomers of 24 to 48 nucleotides and will self-assemble in a single reaction, a “single-pot” synthesis, as described in U.S. Pub. No.: US 2015/0218204, herein incorporated by reference in its entirety. In more preferable embodiments, the canvases are made of 1 to 10,000 nucleotide bricks, from 1 to 1,750 nucleotide bricks, from 1 to 500 nucleotide bricks, or from 1 to 250 nucleotide bricks. In other embodiments, the oligomers comprise of 24 to 42 nucleotides, from 24 to 36 nucleotides, or from 26 to 36 nucleotides.

In another embodiment the architecture is made step wise using a serial fluidic flow to build the final shape as described in U.S. Pat. No. 9,073,962, herein incorporated by reference in its entirety.

In some embodiments, the architecture is assembled using the origami approach. With an origami approach, for example, a long scaffold nucleic acid strand is folded to a predesigned shape through interactions with relatively shorter staple strands. Thus, in some embodiments, a single-stranded nucleic acid for assembly of a nucleic acid nanostructure has a length of at least 500 base pairs, at least 1 kilobase, at least 2 kilobases, at least 3 kilobases, at least 4 kilobases, at least 5 kilobases, at least 6 kilobases, at least 7 kilobases, at least 8 kilobases, at least 9 kilobases, or at least 20 kilobases. In some embodiments, a single-stranded nucleic acid for assembly of a nucleic acid nanostructure has a length of 500 base pairs to 20 kilobases, or more. In some embodiments, a single-stranded nucleic acid for assembly of a nucleic acid nanostructure has a length of 7 to 15 kilobases. In some embodiments, a single-stranded nucleic acid for assembly of a nucleic acid nanostructure comprises the M13 viral genome. In other embodiments, a single-stranded nucleic acid for assembly of a nucleic acid nanostructure comprises an artificial genome. In some embodiments the number of staple strands is less than about 2,000 staple strands, less than about 1,000, less than about 500 staple strands, less than about 400 staple strands, less than about 300 staple strands, less than about 200 staple strands, or less than about 100 staple strands.

In some embodiments, the architecture is assembled from single-stranded tiles (SSTs) (see, e.g., Wei B. et al. Nature 485: 626, 2012, incorporated by reference herein in its entirety) or nucleic acid “bricks” (see, e.g., Ke Y. et al. Science 388:1177, 2012; International Publication Number WO 2014/018675 A1 each of which is incorporated by reference herein in its entirety). For example, single-stranded 2- or 4-domain oligonucleotides self-assemble, through sequence-specific annealing, into two- and/or three-dimensional nanostructures in a predetermined (e.g., predicted) manner. As a result, the position of each oligonucleotide in the nanostructure is known. In this way, a nucleic acid nanostructure may be modified, for example, by adding, removing or replacing oligonucleotides at particular positions. The nanostructure may also be modified, for example, by attachment of moieties, at particular positions. This may be accomplished by using a modified oligonucleotide as a starting material or by modifying a particular oligonucleotide after the nanostructure is formed. Therefore, knowing the position of each of the starting oligonucleotides in the resultant nanostructure provides addressability to the nanostructure.

In some embodiments, the architecture is made from a single stranded oligomer, as described in U.S. Pub. No.: 2018/0044372 and herein incorporated by reference in its entirety. A single strand of DNA used for assembling a nanostructure in accordance with the present disclosure may vary in length. In some embodiments, a single strand of DNA has a length of 500 nucleotides to 10,000 nucleotides, or more. For example, a single strand of DNA may have a length of 500 to 9000 nucleotides, 500 to 8000 nucleotides, 500 to 7000 nucleotides, 500 to 6000 nucleotides, 500 to 5000 nucleotides, 500 to 4000 nucleotides, 500 to 3000 nucleotides, 500 to 2000 nucleotides, 500 to 1000 nucleotides, 1000 to 10000 nucleotides, 1000 to 9000 nucleotides, 1000 to 8000 nucleotides, 1000 to 7000 nucleotides, 1000 to 6000 nucleotides, 1000 to 5000 nucleotides, 1000 to 4000 nucleotides, 1000 to 3000 nucleotides, 1000 to 2000 nucleotides, 2000 to 10000 nucleotides, 2000 to 9000 nucleotides, 2000 to 8000 nucleotides, 2000 to 7000 nucleotides, 2000 to 6000 nucleotides, 2000 to 5000 nucleotides, 2000 to 4000 nucleotides, or 2000 to 3000 nucleotides. In some embodiments, a single strand of DNA may have a length of at least 2000 nucleotides, at least 3000 nucleotides, at least 4000 nucleotides, or at least 5000 nucleotides. In some embodiments, a single strand of DNA may have a length of 500, 600, 700, 800, 900, 1000, 1100, 1200, 1300, 1400, 1500, 1600, 1700, 1800, 1900, 2000, 2100, 2200, 2300, 2400, 2500, 2600, 2700, 2800, 2900, 3100, 3200, 3300, 3400, 3500, 3600, 3700, 3800, 3900, 4100, 4200, 4300, 4400, 4500, 4600, 4700, 4800, 4900, 5100, 5200, 5300, 5400, 5500, 5600, 5700, 5800, 5900, 6600, 6200, 6300, 6400, 6500, 6600, 6700, 6800, 6900, 7100, 7200, 7300, 7400, 7500, 7600, 7700, 7800, 7900, 8100, 8200, 8300, 8400, 8500, 8600, 8700, 8800, 8900, 9100, 9200, 9300, 9400, 9500, 9600, 9700, 9800, 9900, 10000, 50000, or more nucleotides.

In some embodiments, the architecture is two-dimensional and comprises a single layer of bricks or a single scaffold. The single layer of bricks may form a molecular canvas. In other embodiments, the architecture is three-dimensional and may contain 2, 3, 4, 5, 6, 7, 8, 9, 10, 20, 30, or more layers of two-dimensional structures depending on the desired final shape.

In some embodiments, the architecture is attached to a substrate, such as a glass slide, a silicon base, a microfluidics chamber, a breadboard, and/or combinations thereof. In other embodiments, the architecture remains in a solution.

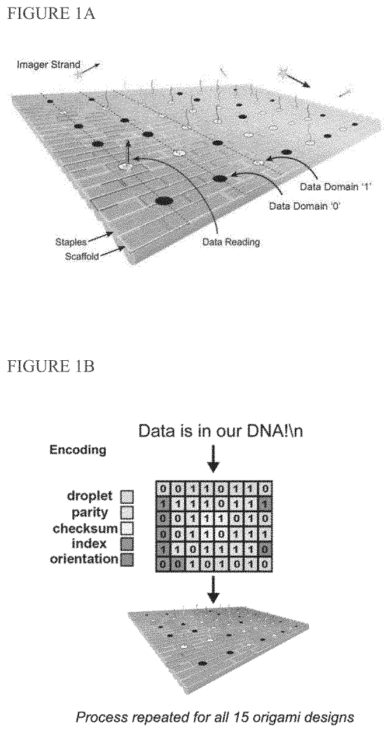

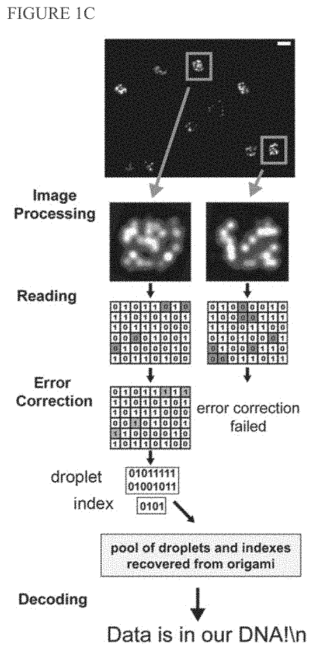

In a preferred embodiment, the architecture is a dNAM origami (FIG. 1A). Preferably, the dNAM origami is a rectangular 2D origami nanostructure formed by staple strands giving a desired shape to a scaffold strand. Specific sequences used to localize data strands to specific sites on the upper surface. This site-specific localization is enabled by extending (1) or not extending (0) the structural staple strands of the nucleic acid origami to create addressable data strands. In other embodiments, the dNAM origami may be stacked into 3D origami nanostructures. Due to the precision of placement by the nucleic acid architecture, the data sites may be placed any distance from each other. The distance between two data sites may be dependent of the resolution capabilities of the microscope and may be, for example, from about 5 nm to about 100 nm, from about 8 nm to about 50 nm, from about 8 nm to about 25 nm, or from about 8 nm to about 15 nm apart. If the data points bring the chromophores to less than about 10 nm apart, they may experience quantum coupling which may cause the two chromophores to act as a single chromophore with different excitation/emission spectra (see U.S. Ser. No. 16/100,052, herein incorporated by reference in its entirety).

The data strands may be evenly positioned within the dNAM architecture or they may be located at specified spots within the dNAM origami. The data strands may have the same docking domains, or the docking domains may be different for one or more data strands. The docking domains may be paired to an imager strand. When the imager strand is paired to the docking domain, the pairing represents a (1) state. For data sites lacking the docking domain, the site represents a (0) state.

The nucleic acid architectures may be stored as appropriate for nucleic acid, such as being refrigerated or frozen in a buffer or lyophilized.

By designing the docking domain of the data strands and image strands to be partially complementary, binding site competition may be used to increase the data density of the compositions. In some embodiments, the docking domain of the data strands are designed to have one or more mismatches to the binding domain of the image strands. In other embodiments, the docking domain of the image strands are designed to have one or more mismatches to the docking domain of the data strands. In yet other embodiments, the docking domains of both the data strand and image strand have been designed to contain mismatched pairs. By designing the docking domains with mismatches, each image and data strand combination will have a unique on/off rate. The unique on/off rate will create a location having a value based on the number of unique sequences that could be resolved temporarily at that location, for example, the value could be 0, 1, 2, or more. This unique on/off rate may be observed temporally so data may be encoded onto the architecture both temporally and spatially at the individual dye level. In some embodiments, data density may be further increased using different and/or multiple dyes on the partially complement data and/or imaging strands, where the different and/or multiple dyes have distinct spectra. This allows for special, temporal, and color to act together to further increase the data density of an architecture.

### Dyes

Using the above architectures, dyes comprising one or more chromophores or fluorophores may be placed in precise locations using the staples making up the data strands. The dyes are bound to the imager strands. In some embodiments, a single dye is bound to an imager strand. In other embodiments, multiple dyes are bound at multiple turns in the imager strand. In some embodiments the dyes are the same within the dNAM origami. In other embodiments, the dyes are multiplexed using orthogonal binding sequences between the docking domain and imager strands utilizing different binding kinetics. Through the use of multiplexing or binding additional dyes to multiple turns of the imager strand, it may be possible to increase the data density of the dNAM origami.

Any dye comprising at least one chromophore may be used in any embodiment. A dye may be symmetrical or asymmetrical and may have additional modifications to change solubility, hydrophobicity, or symmetry in order to adjust the placement of the dye (i.e., its proximity and orientation to another dye or aggregate). By way of non-limiting examples, the dye may be one or more of a xanthene derivatives such as fluorescein, rhodamine, Oregon green, eosin, and Texas red; cyanine derivatives such as cyanine, indocarbocyanine, oxacarbocyanine, thiacarbocyanine, and merocyanine; a squaraine derivative or ring-substituted squaraines such as Seta, SeTau, and Square dyes; a naphthalene derivative such as a dansyl or prodan derivative; a coumarin derivative; a oxadiazole derivative such as pyridyloxazole, nitrobenzoxadiazole and benzoxadiazole; an anthracene derivatives such as anthraquinones including DRAQS, DRAQ7 and CyTRAK Orange; a pyrene derivative such as cascade blue; an oxazine derivative such as Nile red, Nile blue, cresyl violet, oxazine 170; an acridine derivative such as proflavin, acridine orange, acridine yellow; and an arylmethine derivative such as auramine, crystal violet, and malachite green; a tetrapyrrole derivative such as porphyrins, chlorin, porphin, phthalocyanine, and bilirubin; or a dipyrromethene derivative, such as, but not limited to, a BODIPY family dye which have the general formula of C9H7BN2F2, for example 4,4-difluoro-4-bora-3a,4a-diaza-s-indacene. The aggregates may alternatively comprise one or more commercial dye(s), such as but not limited to Freedom™ Dye, Alexa Fluor® Dye, LI-COR IRDyes®, ATTO™ Dyes, Rhodamine Dyes, or WellRED Dyes; or any other dye. Examples of Freedom™ Dyes include 6-FAM, 6-FAM (Fluorescein), Fluorescein dT, Cy3™, TAMRA™, JOE, Cy5™, TAIVIRA, MAX, TET™, Cy5.5™, ROX, TYE™ 563, Yakima Yellow®, HEX, TEX 615, TYE™ 665, TYE 705, and Dyomic Dyes. Examples of Alexa Fluor® Dyes include Alexa Fluor® 488, 532, 546, 647, 660, and 750. Examples of LI-COR IRDyes® include 5′ IRDye® 700, 800, and 800CW. Examples of ATTO™ Dyes include ATTO™ 488, 532, 550, 565, Rhol01, 590, 633, 647N. Examples of Rhodamine Dyes include Rhodamine Green™-X, Rhodamine Red™-X, and 5-TAIVIRA™. Examples of WellRED Dyes include WellRED D4, D3, and D2. Examples of Dyomic Dyes include Dy-530, -547, -547P1, -548, -549, -549P1, -550, -554, -555, -556, -560, -590, -591, -594, -605, -610, -615, -630, -631, -632, -633, -634, -635, -636, -647, -647P1, -648, -648P1, -649, -649P1, -650, -651, -652, -654, -675, -676, -677, -678, -679P1, -680, -681, -682, -700, -701, -703, -704, -705, 730, -731, -732, -734, -749, -749P1, -750, -751, -752, 754, -756, -757, -758, -780, -781, -782, -800, -831, -480XL, -481XL, -485XL, -510XL, -511XL, -520XL, -521XL, -601XL. Examples of other dyes include squaraine, 6-FAM, Fluorescein, Texas Red®-X, and Lightcycler® 640.

NAM and dNAM Architecture

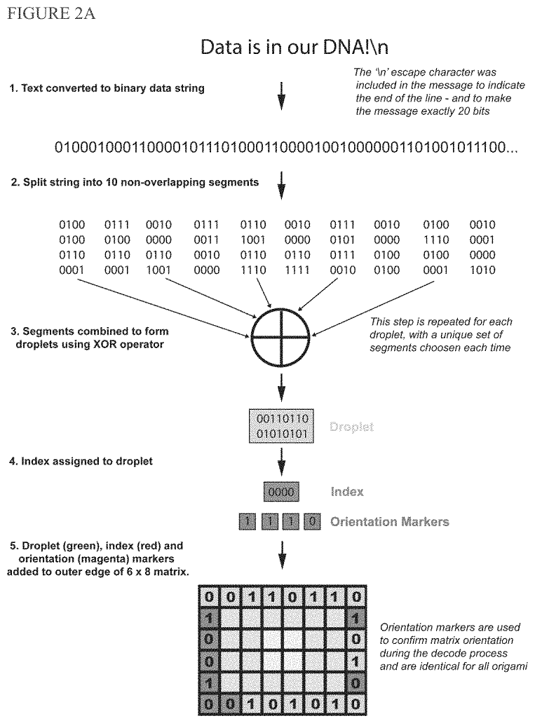

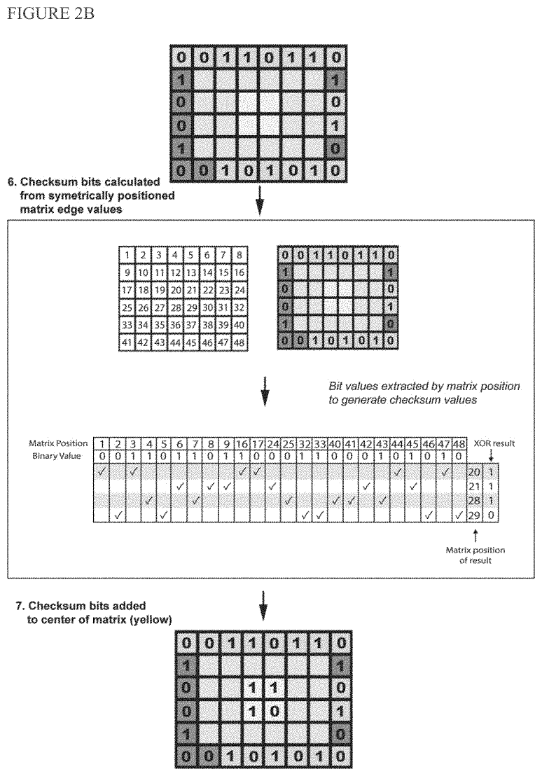

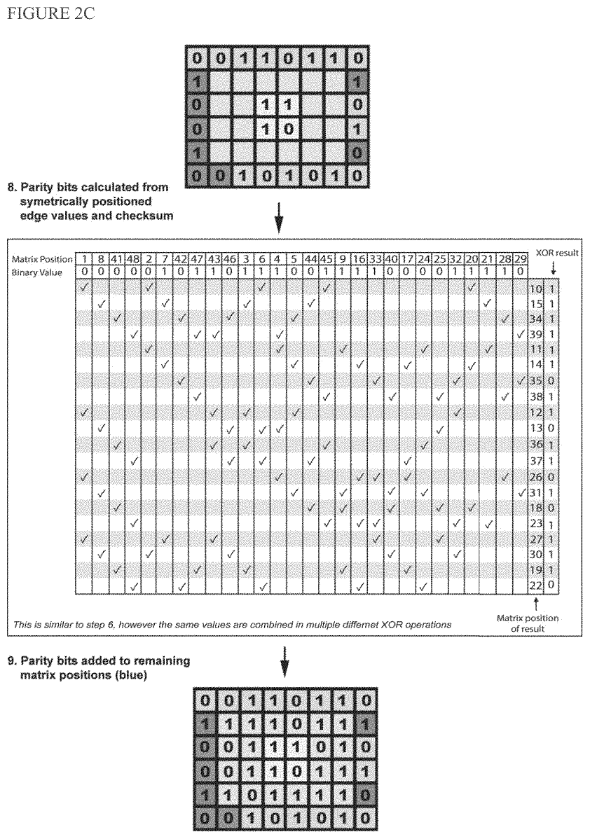

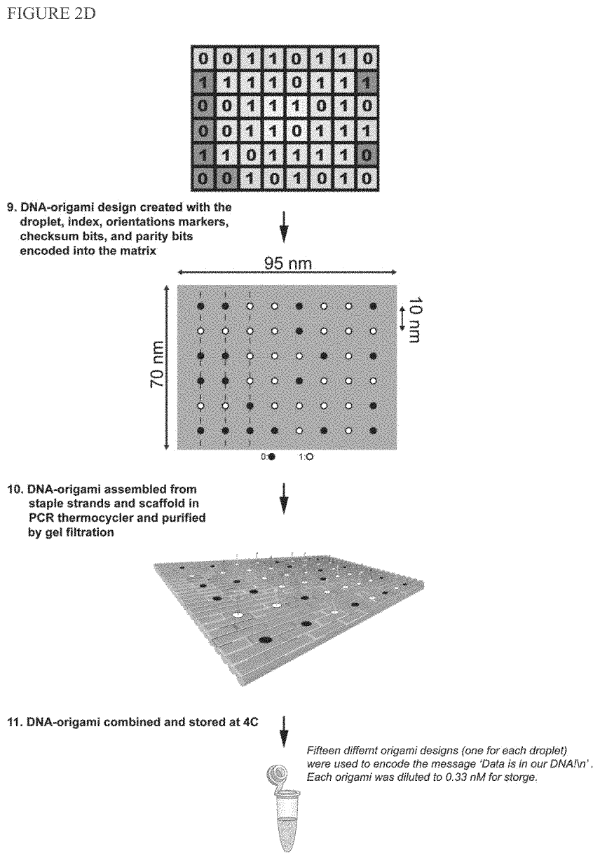

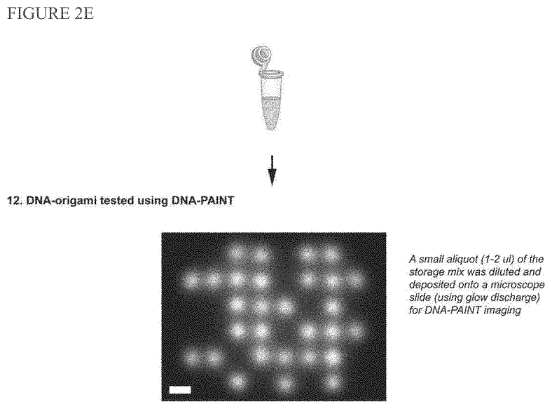

As shown in FIG. 1B and the flowchart in FIGS. 2A to 2C, various bits of data may be encoded within the NAM or dNAM architecture. As described above, each bit is represented by a (1) when the docking domain of a data strand is paired to an image strand and (0) otherwise, such as when the data site is occupied by a staple strand. In some embodiments, the dNAM architecture includes data bits. In some embodiments, the data bits may represent a binary data string. In more preferred embodiments, the binary data string is encoded into one or more distinct architectures or droplets. If the data is encoded into multiple droplets, the dNAM architecture may include a sufficient number of index bits to identify each droplet. In still further embodiments, the dNAM architecture includes error correction, including checksum bits and parity bits. In yet still further embodiments, the dNAM architecture includes orientation markers. In a preferred embodiment, the dNAM architecture includes data bits representing a droplet, index bits, checksum bits, parity bits, and orientation markers.

The actual number of each bit type will depend on the amount of data being saved on the dNAM architecture. The more data that needs to be stored, the larger and/or more complex the dNAM architecture may become.

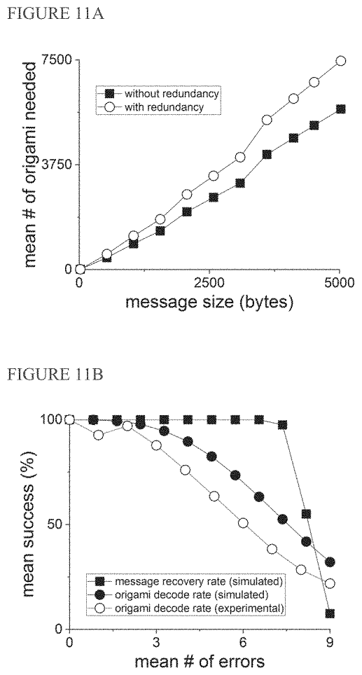

Additionally, as the amount of data increases, the number of dNAM architectures needed to encode the data will also increase. As shown in FIG. 11A, the number of distinct architectures, such as origami, required to encode a message of length n increases roughly at a linear rate up to n=5000 bytes of data. Larger message sizes require more bits to be devoted to indexing, decreasing the number of available data bits per architecture. This limit can be increased, however, by increasing the number of bits per architecture, such as by increasing the size of the architecture to allow for more bits per architecture, using multiple chromophores per image strand, or multiplexing.

Encoding Data onto NAM or dNAM Architecture

The message or data may be stored in the nucleic acid memory as either analog or digital signals. In an aspect, the data is stored in an indexed array. In some embodiments, the message or data is analog and may be stored on the architectures by, for example, positioning the chromophores to write out a text stream or create an image directly with the architectures.

In preferred embodiments, the message or data is stored as digital information, preferably as an indexed array of digital information, represented as bits on the architecture. If the message or data stored is digital, then the NAM is a digital nucleic acid memory (dNAM). As the message or data may be stored on the dNAM architectures as digital bits, any type of message or data may be saved, such as, but not limited to binary, hexadecimal, decimal, octal, text, or graphic. The data may also first be encrypted and/or compressed before storage in a NAM or dNAM. The message or data may be transformed or encoded, for example converting a text message or graphical image data into binary or encoding binary information using a code, such as, but not limited to, fixed rate or rateless codes. Rateless codes may include, but are not limited to, fountain codes, like Luby Transform codes, or spinal codes. In preferred embodiment, the code is a rateless code. In more preferred embodiments, the rateless code is a fountain code. These types of codes allow for the message or data to be stored in a population of dNAM architectures comprising of a number of different members, or droplets, wherein each member has a distinct encoding.

Rateless codes allow for a potentially limitless amounts of encoded bits stored on a population of dNAM architectures to be sent to a receiver, as discussed below, and then to decode the bits back into the corresponding data or message. While a limitless number of encoded architectures, typically only a limited subset need to be created. This limited number will depend on the specific encoding algorithm and the amount of data to be stored. For example, a string of bits may be encoded using a fountain code into any number of distinct NAM architectures, a limited number may then be captured using microscopy, and then decoded. However, for a rateless code to properly function, additional information, such as, but not limited to, error correction bit and index bits, needs to be encoded along with the data to ensure that the limited number of possible distinct architectures received provides a reasonable surety, based on the amount of data stored across the population, that the data has been received (FIG. 2A).

In further embodiments, the dNAM architecture includes error message data bits. These bits may ensure the recovery of the message or data stored within the population of architectures encoded the message. Examples of error message data bits include, but are not limited to, index, parity, checksum, and/or orientation marker bits (FIG. 2A).

In a preferred embodiment the index and orientation bits are assigned to each distinct architecture, with the index bits being unique for each distinct architecture. The index bits are added to the architectures to identify the distinct encoding and architecture. Orientation bits are added to the architectures to confirm the matrix orientation during the decoding process. While any system of orientation bits may be utilized, for example pairing certain orientation bits with certain index bits, in a preferred embodiment the orientation bits are identical across all the architectures (FIG. 2A).

In a further embodiment, at least one error checking set of bits is included within the architecture. For example, checksum bits (FIG. 2B) may be calculated based on message bits, index bits, orientation bits, and/or combinations thereof. In a preferred embodiment, the checksum bits are calculated based on the combination of message bits, index bits, and orientation bits to provide the most amount of information possible within the checksum bits.

In further embodiment, the error checking bits further include parity bits (FIG. 2C). In a preferred embodiment, the parity bits are calculated based on the message bits, index bits, orientation bits, and checksum bits. However, any scheme may be used, for example using subset combinations of the different bits. In a further preferred embodiment, the checksum and parity bits are invariant to the rotation of the architecture, allowing for error checking prior to determining the orientation. This results in a bi-level, parity based, and orientation-invariant error detection scheme which may lead to more robust data recovery.

The bits may be positioned on the architecture in any configuration. In some preferable embodiments, if the architecture is a two-dimensional architecture or origami, the message bits, index bits, and orientation bits are placed along the outer edge with the parity bits placed as a ring within the edge bits, and the checksum bits being placed in the center. In other embodiments, the bits may be randomly placed within the architecture or origami.

In yet other embodiments, due to the flexibility of the bit position and the ability to place dyes in precise locations, it is possible to increase the data density beyond just the specific position of the individual bits. By using more complex data structures, for example linear or non-linear data structures, data may be further encoded into higher level patterns onto the surface of the architecture or origami. By way of nonlimiting example, dyes may be arranged in non-linear, directed or undirected, graph data structures where the dyes may act as the vertices. In other embodiments, the architectures or origamis may be sectioned with each section representing a composite bit. In yet other embodiments, the data on the architecture or origami may be encoded as a barcode or a matrix barcode, such as a Quick Response code (QR code).

More complex encoding may be designed by combining the various embodiments. For example, a matrix barcode may also use mismatched docking domains to create multiple codes in a spatial and temporal manner.

Recovery of Data from Architectures

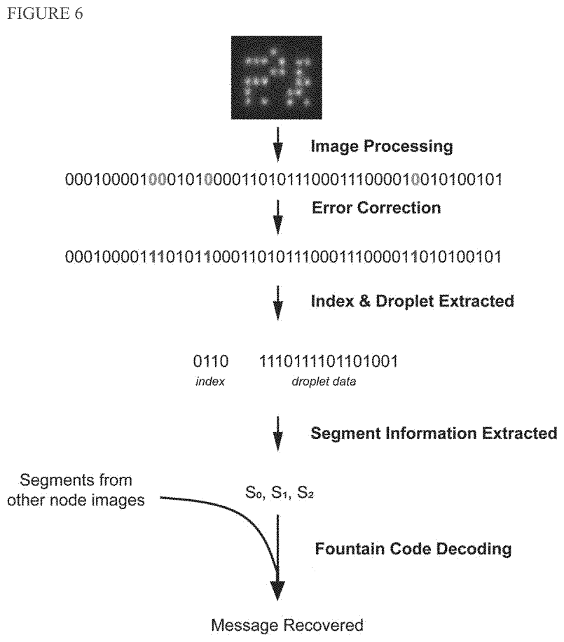

The data may be extracted from the NAM using microscopy that has resolutions which may capture the NAM architecture, for example from about 1 to about 2,000 Å, from about 1 to about 1,500 Å, or from about 1 to about 1,000 Å. For example, super resolution microscopy (SRM), scanning probe microscopy (SPM), atomic force microscopy (AFM), transmission electron cryomicroscopy (cryo-TEM), or single-molecule fluorescence microscopy. In preferred embodiments, any type of fluoresce SRM may be used, including, but not limited to, 4Pi, structured illumination microscopy (SIM), spatially modulated illumination (SMI), spectral precision distance microscopy (SPDM), binding-activated localization microscopy (BALM), photoactive localization microscopy, points accumulation for imaging in nanoscale topography (PAINT), or combinations thereof. In a preferred embodiment, one or more distinct NAM architectures encoding the data to be processed are placed on a cover slip. Using SRM, the cover slip is first imaged at a high enough resolution to capture the distinct patter of chromophores (FIGS. 2E, 3A-B, 5). In a preferred embodiment, the images are captured using PAINT. For dNAM, each chromophore represents (1) while each dark spot represents a (0). While it may be possible to retrieve all the information from a single architecture, in a preferred embodiment, multiple architectures with the same encoding are imaged and averaged together. The single or averaged image is then processed to extract the sequence of bits (FIG. 6).

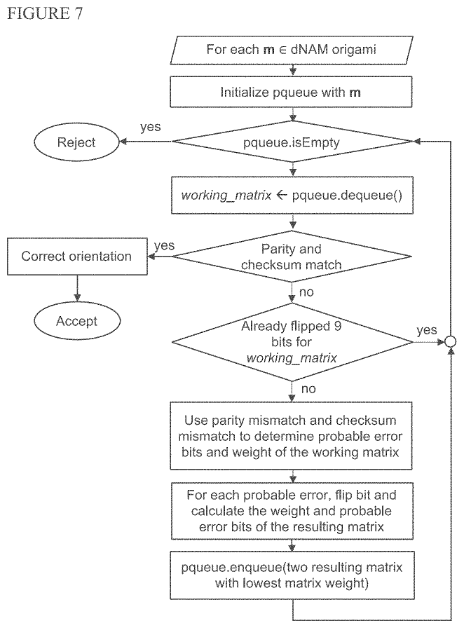

This sequence of bits, if it incorporated error bits, is then checked for errors (FIGS. 6 and 7). FIG. 7 shows an exemplary, preferred error correction flowchart for a single architecture. For example, a priority queue is initialized with an individual architecture, for example a two-dimensional origami, m (the working_matrix). Based on the parity and checksum bits mismatch, the algorithm deduces a set of probable errors and a matrix weight for the working matrix. The matrix weight is proportional to the number of errors, and the main goal of the algorithm is to reduce the matrix weight in a greedy fashion. To that end, each of the probable errors in the working_matrix is sequentially flipped, and a matrix weight calculated for every resulting matrix. The two resulting matrices with the lowest weights are enqueued. The algorithm then replaces the working_matrix with the recalculated matrix possessing the lowest matrix weight from the queue. If the current working matrix already has 9 bits flipped it is discarded and the next matrix in the queue used. The algorithm repeats these steps until the matrix weight equals zero, at this point the data in the origami is considered to have been error-corrected and is passed to the next stage of the decoding (Accept). If the priority queue is emptied before the matrix weight reaches zero, the origami data is considered unrecoverable and is removed from the analysis (Reject).

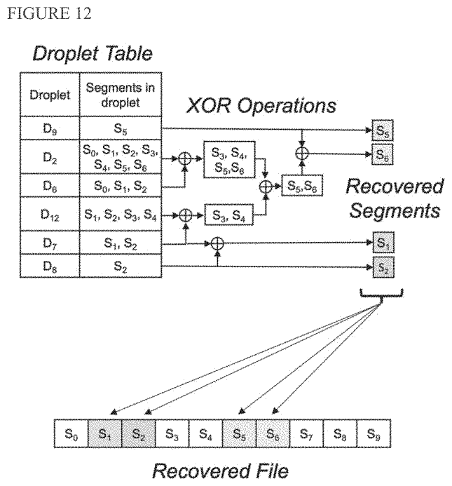

After the bit string has been corrected for any errors, the different segments, such as any message bits or index bits, may then be extracted from the sequence (FIG. 6). Once the message bits are separated from the index and other bits, any appropriate decoding algorithm may then be run. In the preferred embodiment, in which the data is encoded using a fountain code, using a logic operator such as, but not limited to, XOR, into a population of distinct architectures or origami, with at least one architecture imaged in a droplet, the droplets are collected (Droplet Table), with each droplet containing one or more file segments. The data in single degree droplets, such as D9 and D8, encode single segments and are used directly to reconstruct the file (Recovered File). To extract additional individual segment data from multi-segment droplets, the decoding algorithm performs a series of XOR operations. The index information allows the algorithm to determine both the degree of the droplet and which segments of the file that the droplet encodes. Taking the case of D2, a series of XOR operations must be performed in order to retrieve additional segment data from it. The decoding algorithm may XOR a multi-degree droplet with another droplet if the other droplet's segment(s) are a proper subset of the multi-degree droplet. For example, the segments contained in D6 are a proper subset of those in D2. After XORing D2 and D6 a new droplet is generated containing segments S5 and S6, which ultimately leads to the algorithm extracting the data for S6. This process is repeated in a greedy fashion until the algorithm retrieves all of the file's segment data (Recovered File), or it runs out of options for XORing droplets (in which case the entire file cannot be successfully recovered). For simplicity, only six of the 15 possible droplets are shown, with the resulting recovered segments depicted in colored boxes (Recovered Segments; FIG. 12).

In other embodiments, machine learning algorithms may be used to assist in error correction, encoding, and/or decoding the NAM. Machine learning is a branch of artificial intelligence that relates to mathematical models that can learn from, categorize, and make predictions about data. Such mathematical models, which can be referred to as machine-learning models, can classify input data among two or more classes; cluster input data among two or more groups; predict a result based on input data; identify patterns or trends in input data; identify a distribution of input data in a space; or any combination of these. Examples of machine-learning models can include (i) neural networks; (ii) decision trees, such as classification trees and regression trees; (iii) classifiers, such as Naïve bias classifiers, logistic regression classifiers, ridge regression classifiers, random forest classifiers, least absolute shrinkage and selector (LASSO) classifiers, and support vector machines; (iv) clusterers, such as k-means clusterers, mean-shift clusterers, and spectral clusterers; (v) factorizers, such as factorization machines, principal component analyzers and kernel principal component analyzers; and (vi) ensembles or other combinations of machine-learning models. In some examples, neural networks can include deep neural networks, feed-forward neural networks, recurrent neural networks, convolutional neural networks, radial basis function (RBF) neural networks, echo state neural networks, long short-term memory neural networks, transformers, bi-directional recurrent neural networks, gated neural networks, hierarchical recurrent neural networks, stochastic neural networks, modular neural networks, spiking neural networks, dynamic neural networks, cascading neural networks, neuro-fuzzy neural networks, or any combination of these.

Different machine-learning models may be used interchangeably to perform a task. Examples of tasks that can be performed at least partially using machine-learning models include various types of scoring; bioinformatics; cheminformatics; software engineering; fraud detection; customer segmentation; generating online recommendations; adaptive websites; determining customer lifetime value; search engines; placing advertisements in real time or near real time; classifying DNA sequences; affective computing; performing natural language processing and understanding; object recognition and computer vision; robotic locomotion; playing games; optimization and metaheuristics; detecting network intrusions; medical diagnosis and monitoring; or predicting when an asset, such as a machine, will need maintenance.

Any number and combination of tools can be used to create machine-learning models. Examples of tools for creating and managing machine-learning models can include SAS® Enterprise Miner, SAS® Rapid Predictive Modeler, and SAS® Model Manager, SAS Cloud Analytic Services (CAS)®, SAS Viya® of all which are by SAS Institute Inc. of Cary, N.C. Other examples include, but are not limited to, Matlab, scikit-learn, TensorFlow, Weka, Pytorch, Google Cloud AutoML, Azure Machine Learning Studio, IBM Watson, Amazon Machine Learning, Apache Singa, Apache Spark MLLib, Keras, and/or Caffe.

Machine-learning models can be constructed through an at least partially automated (e.g., with little or no human involvement) process called training. During training, input data can be iteratively supplied to a machine-learning model to enable the machine-learning model to identify patterns related to the input data or to identify relationships between the input data and output data. With training, the machine-learning model can be transformed from an untrained state to a trained state. Input data can be split into one or more training sets and one or more validation sets, and the training process may be repeated multiple times. The splitting may follow a k-fold cross-validation rule, a leave-one-out-rule, a leave-p-out rule, or a holdout rule (see U.S. Pat. No. 9,990,367, herein incorporated by reference in its entirety).

For example, a neural network may be taught to predict the outcome of XOR functions and so could replace the XOR steps in the above algorithm. A neural network may also be trained to prioritize bits for the minimum edit distance search in error correction.

Other algorithms may be used depending on the data density and if composite bits were encoded into the architectures. For example, principal component analysis may be used to read regions of the architecture which may represent a composite bit depending on, for example, a certain percentage or locations need an image strand for the composite bit to be a 1. In another example, nearest neighbor algorithms may be used to recover data that has been encoded in a graph data structure onto the architecture.

NAM and dNAM for Stable Data Storage

As NAM and dNAM may be used to store data onto DNA, it provides several benefits over current methods. In one embodiment, NAM or dNAM may be used for stable, long term storage of data, which may first be encrypted and/or compressed. In a further embodiment, NAM or dNAM may be used as backup data storage. In another embodiment, NAM or dNAM may be used for monitoring supply chains by tagging a product or object to prevent counterfeiting. For example, encoding a block chain tracking purchases, manufactures, warehouses, etc. which could then be validated at any point, for example a spot check, along the supply chain. In other embodiments, because data may be stored on the NAM or dNAM, the data may first be encrypted using an algorithm, and then part of the data strands may be used to tag an object to provide physical encryption. The data strands not being stored with the object would act as a key to unlock the encryption. The nucleic acid architecture for data storage as disclosed herein provides several benefits over current methods.

Current long-term storage, used for data that includes, but is not limited to, backup or archival data storage, include hard-disk drives, solid state drives, tape storage, and optical storage. While each of these technologies provide different benefits for backup and archival data storage, they also all have drawbacks. These drawbacks include obsolescence, limited life expectancy, limited data capacity/space considerations, waste generation, and the required maintenance.

For example, most currently available storage options have a limited life expectancy for either the data or the base storage media. Solid state drives tend to lose data over time because as they are used, the passage of electrons through the media can cause leakage. While for hard-disk drives, the magnetism wears off the plates, which can also lead to data loss. This loss requires the drives to be refreshed about every year but will eventually result in failure of the drive. Once a drive wears out it creates electronic waste as it will have to be exposed of. For optical media, rewritable media is unstable compared to the write once media, so there is a balance between creating waste with the write once and have it be available for long-term storage versus being able to refresh the data when needed with the rewritable media. Additionally, the storage media must be kept in dry, climate-controlled rooms to maximize longevity. However, there are already rising concerns about the cooling being used in data centers around the world, including the high energy and water consumption needed. As more data needs to be stored long-term, the energy and water usage will likewise increase and put strains upon available resources.

Obsolescence is also an issue with current technologies. Due to rapid advances in computer technology, it has led to rapid and hard to predict obsolescence. This poses a problem if the backup or archival media selected falls out from being mainstream as finding ways of reading the now obsolete media or repairing the reader may no longer be readily available, and thus driving up the cost of data storage.

These issues are circumvented by NAM. Nucleotides, especially those lacking the 2′ hydroxyl group on the sugar like DNA, have a much higher stability, it is easier to store, and offers more flexibility than current long-term storage media. The stability of nucleotides is much longer than the media currently used. For example, DNA has been extracted from natural samples dating back about 700,000 years and then successfully sequenced. Under more optimal circumstances, DNA stability has been estimated to be in the millions of years. This is significantly more stable than any of the current media being used.

Nucleotides also offer alternate storage capabilities. Current media generally needs to be stored in dry, climate-controlled rooms to prevent damage. Climate-controlled storage generally requires a large amount of energy to run a large air conditioner, which can be a drain on resources and not environmentally friendly. This use of electricity will also increase as more and more data needs to be stored, putting more strain on the use of electricity and the environment. However, given the stability of nucleotides, they may be stored in a variety of environments, including in liquid nitrogen or merely in dry and cool environments. As liquid nitrogen tanks do not require any electricity to keep cold, the storage of nucleotides will cause less of an impact on the environment, providing a surprising benefit over current storage media.

The use of nucleotides as long-term data storage also provides an additional environmental benefit. Current storage media creates a lot of electronic waste due to its lack of stability. The growing amount of electronic waste in the world is of a growing concern due to not only the amount of waste being generated, but also of the harmful or rare compounds sometimes found inside electronic devices. However, nucleotides and proteins are completely biodegradable, and nucleotides may be refurbished more easily then electronic devices due to their properties, such as Watson Crick pairing. Hence, the use of the nucleic acid architectures and chromophores of the instant disclosure are more environmentally friendly than current storage devices.

The storage length of the architectures may be further increased by embedding or encapsulating them in a shell to help protect the nucleotides from interacting with the environment. By way of nonlimiting example, the nucleotides to be stored may be impregnated onto filter paper or encapsulated in a biopolymer or silica nanoparticles. Preferably the nucleotides are encapsulated in silica nanoparticles (for example, see Paunescu, D., et al., Reversible DNA encapsulation in silica to produce ROS-resistant and heat-resistant synthetic DNA ‘fossils’. Nat. Protoc. 8, 2440-2448 (2013), herein incorporated by reference in its entirety). This additional protection extends the types of condition that nucleotides remain stable. For example, it has been shown that nucleotides may remain stable for about 2,000 years at ambient temperatures when recovered from being encapsulated in a silicon shell.

The current disclosure also differs from other nucleotide storage systems, methods, and constructs. Other nucleotide storage systems are akin to the inherent properties of DNA to encode protein information. These systems first assign one or more bases to represent a symbol, such as a binary (1) or (0) or if text A through Z. The systems them encode the data onto one or more strand of nucleotides. To recover the data, the one or more strands are sequenced, and if necessary, assembled and decoded back into the data. Hence, in these systems, the data corresponds directly to the sequence of the strand. However, in this disclosure, the strand sequence is only used to form an architecture and is independent of the data. The positioning of the chromophores in an indexed array is what corresponds to the data being stored.

The use of a population of strands to create an architecture also permits an added layer of encryption. By distributing the data onto data strands, the data may be physically separated from the other structural strands, for example the scaffold strand in origami or the other staples or bricks that may allow the self-assembly of the full architecture. Hence, without knowing the proper sequence to allow assembly of the architecture, even if all the data strands were known, the data could not be retrieved without the additional structural sequences. Therefore, as both the data may be encrypted and the strands physically separated, the compositions disclosed herein offer both physical and algorithmic encryption.

Further, given the size of nucleic acids and dyes, the ability to separate the population of strands, the biocompatibility, and the information density of an architecture, the compositions may be miniaturized to such an extent that they may be placed in other various compositions. For example, a portion, such as just the data strands or all the staples, or the entire architecture may be mixed into any product, such as, but not limited to, a pharmaceutical, nutraceutical, paint, glue, powder, food, detergent. The data that may be encoded may include information for tracking the product. More specifically, the data may include the origin, manufacturer, distributor, or recipient of the product.

The NAM and dNAM may also be used to label products which have met regulatory approval. For example, a product, such as pharmaceutical, pesticide, herbicide, or genetically modified organism, that requires regulatory approval may have a specific regulatory agency's information encoded onto an architecture and then the architecture or the data strands may be included within the product. The type of information that may be stored may include a data string to identify the regulator and any additional information, such as under which regulation, the product was verified. A QR code, blockchain, or an image, similar to a watermark, may also be encoded onto the architecture to identify approval.

The compositions disclosed herein also differ from the currently used nucleotide strands in what needs to be stored. In current methods, the data is encoded onto the nucleotide string, and then the string is generally included in an expression vector before storage. Hence, current methods store long nucleotide constructs. However, in an embodiment of the current disclosure, only the data strands are stored, wherein the nucleic acid architecture is an origami. In another embodiment, only the data strands comprising a docking domain are stored, wherein the nucleic acid architecture is an origami. As the data strands with the docking domains are what bind to the imager strands, which in turn are bound to the chromophore, and for the cases of origami, the scaffold strand may be known and held constant. Hence, only the data strands which will bind to chromophores need to be stored. However, in other embodiments, all the staple strands are stored, and in yet further embodiments, the scaffold strand and the data strands are stored. Similarly, for the nucleotide brick molecular canvases or single-stranded tiles, all the bricks or tiles may be stored or just those bricks or tiles with a docking domain may be stored. For single stranded oligomers, the oligomer is stored. Therefore, the systems, methods, and compounds disclosed herein differ from other forms of nucleotide storage.

NAM and dNAM for Temporary or Short-Term Data Storage

While the architecture or subsets of the architectures may be used for long term storage, the architectures may also be used for temporary or short-term data storage. For example, due to the heat sensitive nature of base pairing, a fully assembled architecture may be placed into heat sensitive products to detect if the product has been exposed to elevated heat, chemical degradation, or ultra-violet light. The elevated heat or ultra-violet light may cause part or all the architecture to denature and so when imaged, the partially or fully denatured architecture will provide a partial restoration or a blank image. Additionally, this may also be used to identify products that have been sterilized as sterilization will cause the architecture to denature. The degradation of the nucleic acids may also be used to identify the age of a product. If the architecture or part of an architecture is placed within a product and is unprotected, it will degrade. By adding a sufficiently large quantity of an architecture or a part of an architecture, the degradation may be used to correspond to a use by or best by date. Further, as the different sugars that make up the nucleotides, for example ribose or deoxyribose, degrade at different rates, architectures storing the same information but having different sugars may be designed to degrade a certain amount of one architecture before a second having a different sugar.

Additional modification to the sugars, such as by adding or removing reactive groups or by locking or bridging the sugar may also affect the degradation of the nucleotides. For example, LNA and BNA increase duplex stability, which would increase the denaturing temperature, and protect the nucleotides from nucleases. Conversely, UNA decreases duplex stability allowing for lower denaturing temperatures of an architecture.

Therefore, by altering the sugar of the nucleotides it is possible to tune the stability of either specific regions of the architecture or the entire architecture, allowing for its use not only for long term, stable storage, but also for short term, temporary storage and identification.

NAM and dNAM Systems for Stable Storage

Systems for encoding digital information into indexed arrays can comprise systems, methods, and devices for converting files and data (e.g., raw data, compressed zip files, integer data, and other forms of data) into bytes or bits and encoding the bytes or bits into segments or sequences of nucleic acids, typically DNA, or combinations thereof.

In an aspect, the present disclosure provides systems for encoding data of any kind using indexed arrays comprising of a nucleic acid architecture and, for super-resolution microcopy, dyes. In an embodiment, a system for encoding binary data using indexed arrays comprising of a nucleic acid architecture and chromophores may comprise a device and one or more computer processors. The device may be configured to synthesize a nucleic acid architecture or data strands comprising docking domains. The one or more computer processors may be individually or collectively programmed to (i) encode the data into a binary sting, (ii) select data strands comprising docking domains, and (iii) construct an indexed array of the binary string.

Depending on the amount of the data being stored, the one or more computer processors, in further embodiments, may be individually or collectively programmed to further perform one or more additional tasks, such as, but not limited to: (i) create a rateless code, (ii) create index bits and translate into the binary string, (iii) create orientation bits and translate into the binary string, (iv) calculate parity bits and translate into the binary string, and/or (v) calculate checksum bits and translate into the binary string.

In another aspect, the present disclosure provides systems for reading data from the indexed arrays. In one embodiment, a system for reading data from the indexed arrays may comprise a microscope and one or more computer processors. The microscope may identify the status, for example a (1) or a (0) for binary data, of the data strands on the nucleic acid architecture by detecting if the data strand is bound to an imager strand through the excitation of a chromophore. The one or more computer processors may be individually or collectively programmed to (i) capture the image from the microscope, (ii) identify the status of the data strands, (iii) generate a plurality of symbols from the data strands in (ii), and (iv) compile the information from the plurality of symbols.

Non-limiting embodiments of methods for using the system to encode or recover data are described above.

Any nucleotide synthesis device may be used to make the different strands required to form the nucleic acid architecture. Various nucleotide synthesis devises are known in the art. The nucleotide synthesis device should be selected to ensure sufficient length of an oligomer may be synthesized. For example, a Kilobase machine is limited to oligomers that are 200 bases or shorter. While this may be used to make bricks or staples, it is insufficient to make long single stranded molecules for single stranded oligomer-based architectures or for the scaffold strands for origami. Similarly, any light microscopy, super resolution microscopy (SRM), scanning probe microscopy (SPM), atomic force microscopy (AFM), transmission electron cryomicroscopy (cryo-TEM), or single-molecule fluorescence microscope, such as those available from Leica, or Nikon, may be used.

Information storage in indexed arrays of nucleic acid architectures may have various applications including, but not limited to, long term information storage and sensitive information storage, such as archival storage of medical, genealogical, or financial information.

Computer Systems Using NAM or dNAM

The present disclosure provides computer systems that are programmed to implement methods of the disclosure. For example, a computer system may be programmed or otherwise configured to encode digital information into indexed arrays comprising a nucleic acid architecture and chromophores and/or read (e.g., decode) information derived from indexed arrays comprising a nucleic acid architecture and chromophores. The computer system can regulate various aspects of the encoding and decoding procedures of the present disclosure, such as, for example, the bit-values and bit location information for a given bit or byte from an encoded bitstream or byte stream.

The exemplary computer system includes a central processing unit (CPU, also “processor” and “computer processor” herein), which can be a single core or multi core processor, or a plurality of processors for parallel processing. The computer system may also include additional components, such as, but not limited to, memory or memory location (e.g., random-access memory, read-only memory, flash memory), electronic storage unit (e.g., hard disk), communication interface (e.g., network adapter) for communicating with one or more other systems, and peripheral devices, such as cache, other memory, data storage, and/or electronic display adapters. The memory, storage unit, interface and peripheral devices are in communication with the CPU through a communication bus, such as a motherboard. The storage unit can be a data storage unit (or data repository) for storing data. The computer system may be operatively coupled to a computer network (“network”) with the aid of the communication interface. The network may be the Internet, an internet and/or extranet, or an intranet and/or extranet that is in communication with the Internet. The network in some cases is a telecommunication and/or data network. The network may include one or more computer servers, which can enable distributed computing, such as cloud computing. The network, in some cases with the aid of the computer system, can implement a peer-to-peer network, which may enable devices coupled to the computer system to behave as a client or a server.

The CPU may execute a sequence of machine-readable instructions, which can be embodied in a program or software. The instructions may be stored in a memory location, such as the memory. The instructions can be directed to the CPU, which can subsequently program or otherwise configure the CPU to implement methods of the present disclosure. Examples of operations performed by the CPU can include fetch, decode, execute, and writeback.

The CPU can be part of a circuit, such as an integrated circuit. One or more other components of the system may be included in the circuit. In some cases, the circuit is an application specific integrated circuit (ASIC).

The storage unit may store files, such as drivers, libraries and saved programs. The storage unit may store user data, e.g., user preferences and user programs. The computer system in some cases can include one or more additional data storage units that are external to the computer system, such as located on a remote server that is in communication with the computer system through an intranet or the Internet.

The computer system may communicate with one or more remote computer systems through the network. For instance, the computer system can communicate with a remote computer system of a user or other devices and or machinery that may be used by the user in the course of analyzing data encoded or decoded in an indexed array. Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system via the network.

Methods as described herein may be implemented by way of machine (e.g., computer processor) executable code stored on an electronic storage location of the computer system, such as, for example, on the memory or electronic storage unit. The machine executable or machine-readable code can be provided in the form of software. During use, the code can be executed by the processor. In some cases, the code can be retrieved from the storage unit and stored on the memory for ready access by the processor. In some situations, the electronic storage unit can be precluded, and machine-executable instructions are stored on memory.

The code can be pre-compiled and configured for use with a machine having a processer adapted to execute the code or can be compiled during runtime. The code can be supplied in a programming language that can be selected to enable the code to execute in a precompiled or as-compiled fashion.

Aspects of the systems and methods provided herein, such as the computer system, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical, and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system can include or be in communication with an electronic display that comprises a user interface (UI) for providing, for example, sequence output data including chromatographs, sequences as well as bits, bytes, or bit streams encoded by or read by a machine or computer system that is encoding or decoding nucleic acids, raw data, files and compressed or decompressed zip files to be encoded or decoded into an index matrix comprising a nucleic acid architecture. Examples of UI's include, without limitation, a graphical user interface (GUI) and web-based user interface.

Methods and systems of the present disclosure can be implemented by way of one or more algorithms. An algorithm can be implemented by way of software upon execution by the central processing unit. The algorithm can, for example, be used with raw data or zip file compressed or decompressed data, to determine a customized method for coding digital information from the raw data or zip file compressed data, prior to encoding the digital information.

The invention is further described in detail by reference to the following experimental examples. These examples are provided for purposes of illustration only and are not intended to be limiting unless otherwise specified. Thus, the invention should in no way be construed as being limited to the following examples, but rather, should be construed to encompass any and all variations which become evident as a result of the teaching provided herein.

### EXAMPLES

Embodiments of the present invention are further defined in the following non-limiting Examples. It should be understood that these Examples, while indicating certain embodiments of the invention, are given by way of illustration only. From the above discussion and these Examples, one skilled in the art can ascertain the essential characteristics of this invention, and without departing from the spirit and scope thereof, can make various changes and modifications of the embodiments of the invention to adapt it to various usages and conditions. Thus, various modifications of the embodiments of the invention, in addition to those shown and described herein, will be apparent to those skilled in the art from the foregoing description. Such modifications are also intended to fall within the scope of the appended claims.

### Example 1

We report digital Nucleic Acid Memory (dNAM), a novel approach to DNA-based data storage. In dNAM, data is encoded by selecting specific combinations of single-stranded DNA possessing (1) or lacking (0) docking site domains. When combined with scaffold DNA these staple strands form DNA-origami optical breadboards from which data is read by monitoring binding of fluorescent imager probes using DNA-PAINT super-resolution microscopy. To enhance data retention, we created a multi-layer error correction scheme that combines fountain codes with bi-level parity codes. As a prototype, 15 origami were encoded with ‘Data is in our DNA!\n’, with each origami encoding a unique data droplet. Our error-correction algorithms ensured that we recovered 100% of the message even when individual docking sites, or entire origami, were missing. Unlike other DNA-based data storage systems, reading dNAM does not require sequencing. As such, it offers a new pathway to harness the advantages of DNA as an emerging memory material.

### Introduction

As outlined by the Semiconductor Research Corporation, archival memory materials are quickly approaching their physical and economic limits^{1,2}. Motivated by the rapid growth of the global datasphere³, and its environmental impacts, new non-volatile memory materials are needed. As a sustainable alternative, DNA is a viable option because of its vast information density, significant retention time, and low energy of operation⁴. While synthesis and sequencing cost curves drive innovations in the field⁵, divergent approaches to nucleic acid memory (NAM) have been limited by the focus on using sequencing to recover stored digital information^{6,7,8,9,10,11,12,13,14.}

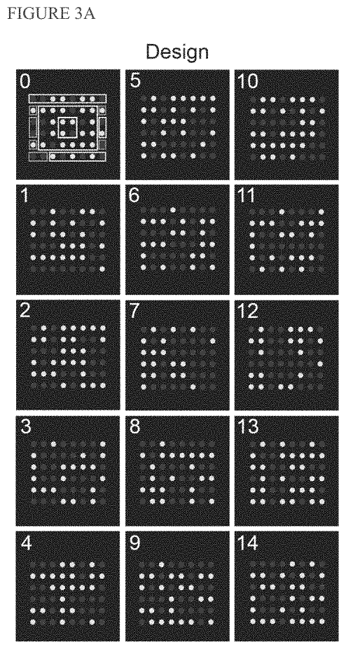

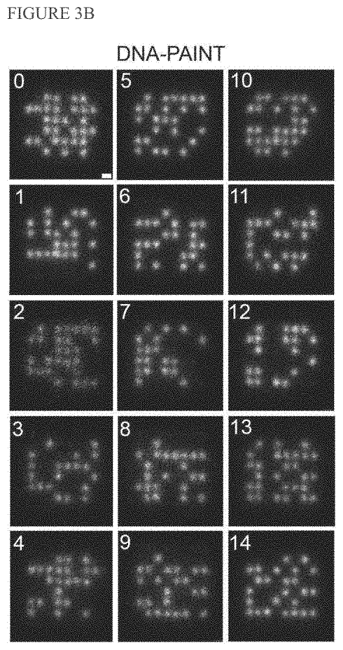

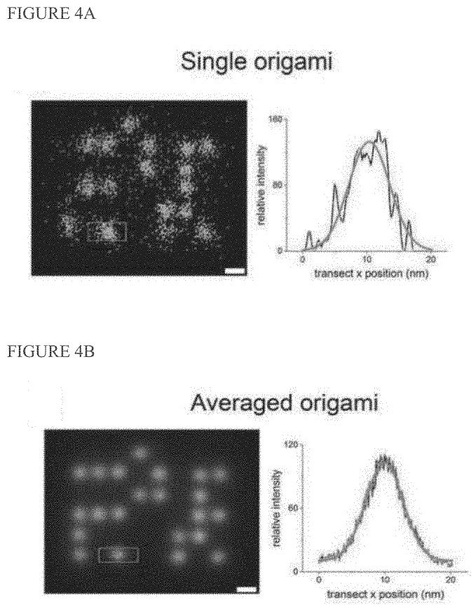

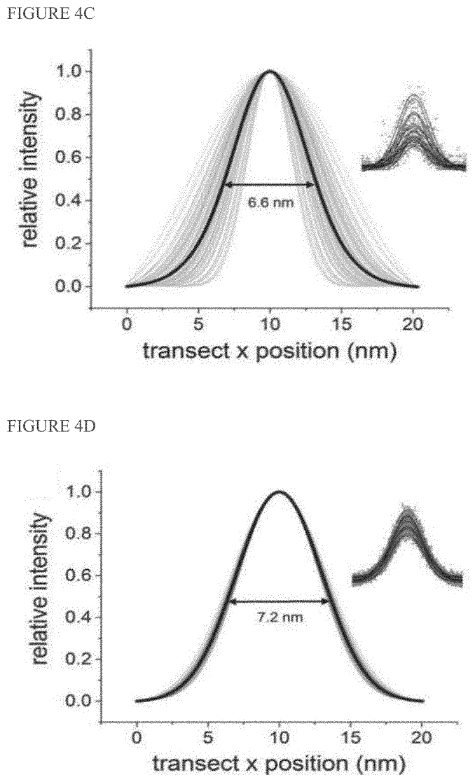

Here, we report an alternative approach to DNA memory via the creation of digital nucleic acid memory (dNAM)—which is inspired by innovations in DNA nanotechnology¹⁵ and made possible by recent advancements in super-resolution microscopy (SRM)¹⁶. In dNAM, non-volatile information is digitally encoded into specific combinations of single-stranded DNA, commonly known as staple strands, that can form DNA origami nanostructures when combined with a scaffold strand. When formed into DNA origami, the staple strands are arranged at addressable locations (FIGS. 1A-1C) that define an indexed array of digital information. This site-specific localization of digital information is enabled by designing staple strands with nucleotides that extend from the origami. Extended staple strands have two domains: the first domain forms a sequence-specific double helix with the scaffold and determines the address of the data within the origami; the second domain extends above the origami and, if present, provides a docking site for fluorescently labelled single-stranded DNA imager strands. Binary states are defined by the presence (1) or absence (0) of the data domain, which is read with a super-resolution microscopy technique called DNA-Points Accumulation for Imaging in Nanoscale Topography (DNA-PAINT)¹⁷. Unique patterns of binary data are encoded by selecting which staple strands have and do not have data domains. As an integrated memory platform, data is entered into dNAM when the staple strands encoding 1 or 0 are selected for each addressable site. The staple strands are then stored directly, or self-assembled into DNA-origami and stored. Editing data is achieved by replacing specific strands or the entire content of a stored structure. To read the data, the origami are optically imaged below the diffraction limit of light using DNA-PAINT (FIGS. 4A-4D).

Key design features of dNAM, that ensure error-free data recovery, are our error-correcting algorithms. Detection of individual DNA molecules using DNA-PAINT is routinely limited by incomplete staple strand incorporation, defective imager strands, fluorophore bleaching, and background fluorescence¹⁸. Although it is possible to improve the signal-to-noise ratio by averaging multiple images of identical structures¹⁸, this approach comes at a significant cost to the read speed and information density. To overcome these challenges, we created dNAM-specific information encoding and decoding algorithms that combine fountain codes with a custom, bi-level, parity-based, and orientation-invariant error detection scheme. Fountain codes enable transmission of data over noisy channels¹⁹. They work by dividing a data file into smaller units called droplets and then sending the droplets at random to a receiver. Droplets can be read in any order and still be decoded to recover the original file²⁰, so long as a sufficient number of droplets are sent to ensure that the entire file is received. We encode each droplet onto a single origami and add additional bits of information for error correction to ensure that individual droplets will be recovered, in the presence of high noise, from individual origami. Together, the error correction and fountain codes increase the probability that the message is fully recovered while minimizing the number of DNA origami that must be observed.

In this report, we describe the first working prototype of dNAM. As a proof of concept, we encoded the message ‘Data is in our DNA!\n’ into origami and recovered the message using DNA-PAINT. We divided the message into 15 digital droplets, each encoded by a separately synthesized origami with addressable staple strands that space data domains approximately 10 nm apart. A single DNA-PAINT recording recovered the message from 20 femtomoles of origami, with approximately 750 origami needing to be read to reach a 100% probability of full data retrieval. By combining the spatial control of DNA nanotechnology with our error correction algorithms, we demonstrate dNAM as a massively parallel optical technology for archival memory applications.

### Results

Recovery of a Message Encoded into dNAM

To test our dNAM concept, we encoded the message ‘Data is in our DNA!\n’ into 15 distinct DNA-origami nanostructures (FIG. 1A). Each origami was designed with a unique 6×8 data matrix that was generated by our encoding algorithm with data domains positioned ˜10 nm apart. For encoding purposes, the message was converted to binary code (ASCII) and then segmented into 15 overlapping data droplets that were each 16 bits. Inspired in part by digital encoding formats like QR-codes, the 48 addressable sites on each origami were used to encode one of the 16-bit data droplets, as well as information used to ensure the recovery of each data droplet. Specifically, each origami was designed to contain a 4-bit binary index (0000 -1110), twenty bits for parity checks, four bits for checksums, and four bits allocated as orientation markers (FIG. 1B). To fully recover the encoded message, we synthesized each origami separately, deposited an approximately equal mixture of all 15 designs (˜20 femtomoles of total origami) onto a glass coverslip, and recorded 40,000 frames from a single field of view using DNA-PAINT (˜4500 origami identified in 2,982 μm²). Super-resolution images of the hybridized imager strands were reconstructed from signal blinks identified in the recording to map the positions of the data domains on each origami (FIG. 1C). Using a custom localization processing algorithm, the signals were translated to a 6×8 grid and converted back to a 48-bit binary string—which was passed to the decoding algorithm for error correction, droplet recovery, and message reconstruction. The process enabled successful recovery of the dNAM encoded message from a single super-resolution recording.

Quality Control of dNAM

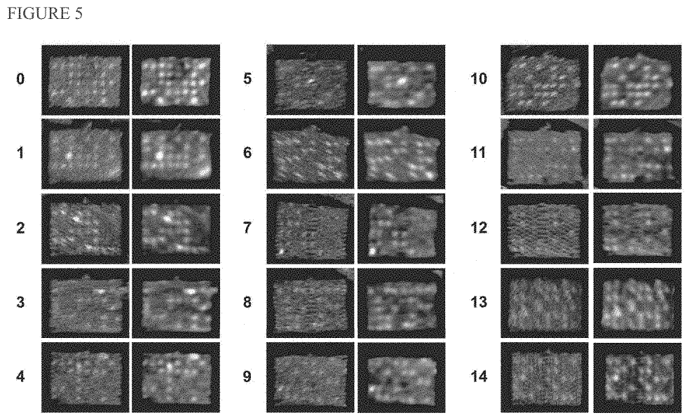

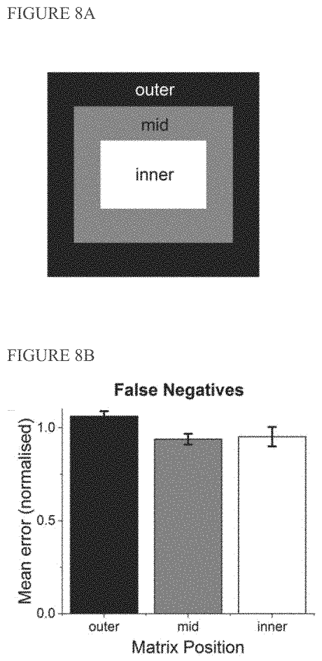

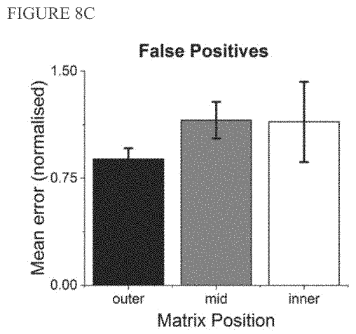

We evaluated all of the origami structures in order to confirm that the 15 different designs were successfully synthesized, with data domains in the intended addresses. Automated image processing algorithms were developed to identify, orient and average multiple images of each origami from the DNA-PAINT recording of the mixture (FIGS. 3A-3B). Although the edges of origami were more sensitive to data strand insertion failures (FIGS. 8A-8C), the results confirmed that all of the data domains, in each of the origami designs, were detectable in each of three separate experiments. Each individual origami synthesis was visualized and validated by atomic force microscopy (AFM). The AFM images further confirmed that the general shapes of all 15 origami designs were as expected with properly positioned data domains (FIG. 5). The results indicate that the extended staple strands do not substantially inhibit the synthesis of the 15 unique origami designs.

Further AFM Analysis of dNAM Origami

As an additional quality control step, we also used AFM to examine origami deposited onto a glass coverslip immediately following SRM imaging. We were not able to resolve individual docking sites in these images, most likely due to the increased roughness of glass, as compared to mica. However, it was possible to count the number of origami in a field of view for comparison with SRM. The densities of origami estimated from the images were 2.4 and 1.4 origami/μm² for AFM and SRM respectively, suggesting that ˜60% of the total origami deposited have their docking sites facing away from the coverslip and available for imager strand binding. To further investigate the variance in error rates between origami designs, we resynthesized the most error prone origami (origami-2). DNA-PAINT imaging indicated that the fresh original batch showed 9.7±2 false negative errors per origami, consistent with the original experiment, while the second batch showed 7.1±2 false negative errors. This suggests that at least a portion of the variance in error rates is independent of origami design and may be caused by variations in mixing, folding, and purification conditions.

Data Encoding/Decoding Strategy for dNAM

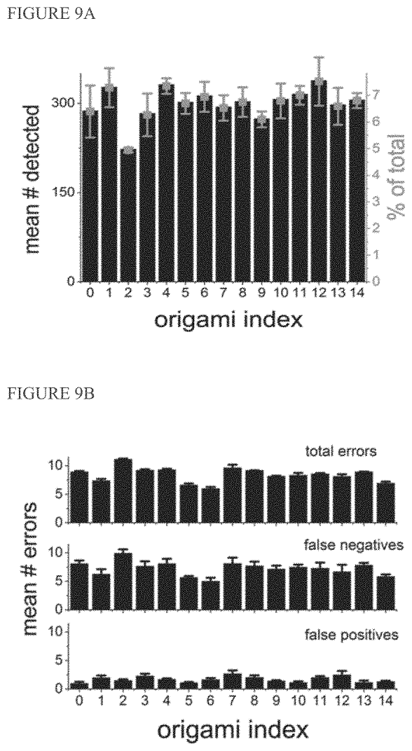

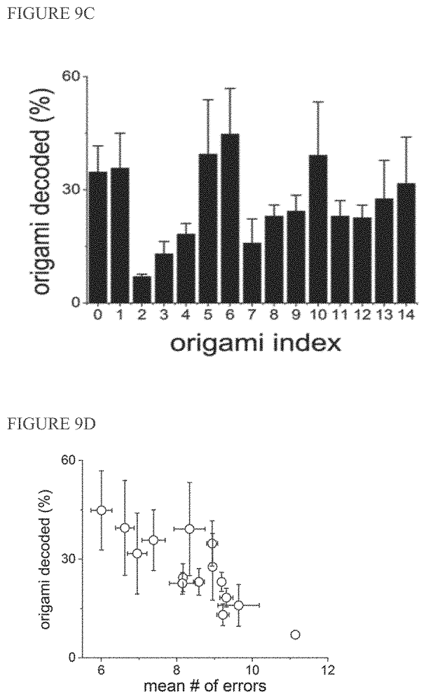

Our encoding approach added 24 error-correction bits of data to every origami structure so that data droplets can be determined from individual origami even when data domains are incorrectly resolved, and the entire message recovered if some droplets are missed entirely. To evaluate the performance of the decoding algorithm, we examined the frequency and types of errors in the DNA-PAINT images and the effect of these errors on our decoding outcomes. We used a template matching strategy where each of the 15 origami grid designs were considered a template, and each individual origami in the field of view was compared to these designs to find a best match. We identified the total number of origami that matched, or did not match, each design (FIGS. 9A-9B). We then determined the number of each design identified by the decoding algorithm when recovering the message (FIG. 9C)—a process independent of template matching and blind to the droplet data contained in the DNA origami. We observed a clear negative correlation between the number of errors detected in a specific design and the number of corresponding data droplets that were successfully decoded by the algorithm (FIG. 9D). The results indicate that, even though there was a low relative abundance of several origami in the deposition mixture (particularly origami-2) and a mean false negative rate of 7.3±1.2% across the different designs, our error-correction scheme enabled successful message recovery. False positives were much less common in our experiments, with a mean of 1.7±0.5% (FIG. 9B). Furthermore, the mean number of errors overcome by the decoding algorithm (5.5±0.1) was lower than the mean number of errors observed across all the origami (7.7±0.1), demonstrating the challenge of decoding origami when several fluorescent signals are missing (FIG. 9E). Nevertheless, the ability of our data encoding and decoding strategy to recover the message despite errors in individual origami is very promising, and the results provide useful guidelines for evaluating and optimizing origami performance for future dNAM designs.

Sampling Analysis of dNAM

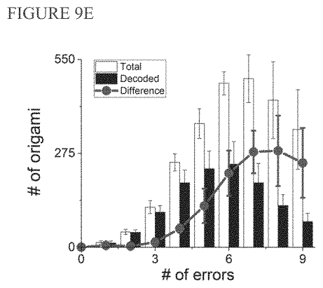

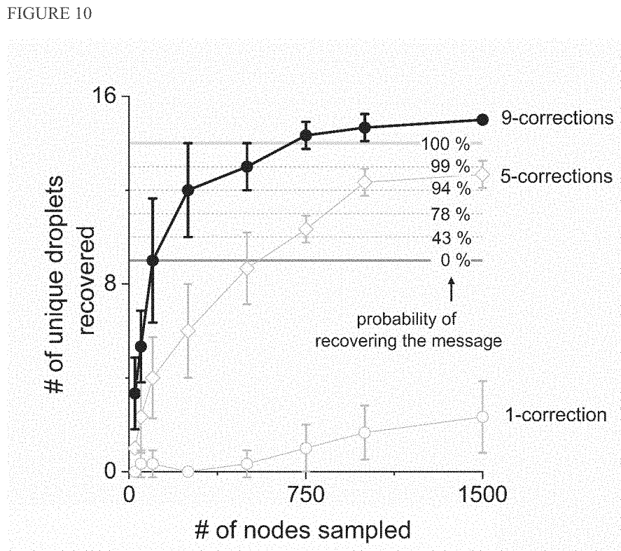

Given the observed frequency of missing data points, we used a random sampling approach to determine the number of origami needed to decode the ‘Data is in our DNA!\n’ message under our experimental conditions. We started with all the decoded binary output strings that were obtained from the single-field-of-view recordings and took random subsamples of 50-3000 binary strings. We passed each random subsample of strings through the decoding algorithm and determined the number of droplets that were recovered (FIG. 10). Based on the algorithmic settings used in the experiment, we found that only ˜750 successfully decoded origami were needed to recover the message with near 100% probability. This number is largely driven by the presence of origami in our sample that were prone to high error rates and thus rarely decoded correctly (i.e., origami-2).

Simulations of dNAM

Simulations were run to determine the size efficiency of the encoding scheme, as well as its ability to recover from errors. As shown in FIG. 11A, the number of origami required to encode a message of length n increases roughly at a linear rate up to n=5000 bytes of data. Larger message sizes require more bits to be devoted to indexing, decreasing the number of available data bits per origami, creating a practical limit of 64 kilobytes of data for the prototype described in this work. This limit can be increased, however, by increasing the number of bits per origami. To determine the ability of the decoding and error correction algorithm to recover information in the presence of increasing error rates, in silico origami that encoded randomly generated data, were subjected to increasing bit error rates. The decoding algorithm robustly recovers the entire message for all tested message sizes when the average number of errors per origami is less than 7.4 (FIG. 11B). At 7.4 errors per origami, the message recovery rate drops to 97.5%, and as expected decreases rapidly with higher error rates (55% recovery at 8.2 errors per origami, and 7.5% at 9 errors per origami). An important feature of our algorithm is that the origami recovery rate can be very low (as low as 63%) and still recover the entire message 100% of the time.

### Discussion

Our results demonstrate a proof of concept for writing, editing, storing and reading of digital information encoded in DNA origami structures. Because of the durability of DNA, dNAM is well suited for archival information storage. Currently, the most widely used material for this purpose is magnetic tape. Recent advancements in magnetic tape report a two-dimensional areal information density up to 31 Gbit/cm^{2,21} though the current commercially available material typically has lower density⁹. Although relevant only for reading throughput, not storage, the information density of tape can be compared to the dNAM origami, which contain data domains spaced at 10 nm intervals to achieve an areal density of about 1000 Gbit/cm². Even after accounting for using ˜2/3 of the bits for indexing and error correction, this still results in an areal data density of 330 Gbit/cm². It is possible to increase dNAM areal density by placing a data domain at every turn in the DNA helix (˜3.5 nm spacing), a distance that has been resolved by SRM²². Other avenues to increasing density are also available, such as previously reported multiplexing techniques with multiple fluorophores and orthogonal binding sequences with different binding kinetics³³, and incorporation of each of these approaches is expected to impact reading throughput. In terms of durability, typical magnetic tape lasts for 10-30 years, while double stranded DNA is estimated to be stable for millions of years under optimal environmental conditions₈.

With our current microscope setup and origami deposition protocol we can image the 7,500 unique origami designs needed to store 5 kB of data (FIG. 5), albeit in several recordings. We conservatively estimate it would take ˜30 recordings to ensure a 100% probability of successful data recovery given the error rates we observed. While it is possible to use dNAM, as described here, to store up to 64 kB the number of origami designs required to meet the increased indexing demands make this impractical. To efficiently handle larger datasets, it is necessary to improve the indexing capacity of individual origami. This could be achieved by engineering larger origami or by simply increasing data density—either by placing data sites closer together or by using multiplexing techniques to augment bit depth at each site. Improvements in read speed could be achieved by depositing origami at higher concentrations, making simultaneous recordings, and by optimizing dNAM to work with shorter, faster binding, imager strands. Our previous work²⁴ shows close-packing of origami is possible on boron-implanted silicon substrates, demonstrating a potential route forward for reducing reading times.

Our results also indicate that advancements in origami-based information storage and reading will require a coordinated effort between improvements in origami synthesis, substrate deposition, DNA-PAINT, and coding algorithms. For example, our subsampling approach (FIG. 10) showed that a decoding algorithm that corrected up to nine errors easily recovered our entire message, while algorithms that corrected only five or fewer errors are much less computationally expensive but rarely recovered our full message. This makes sense, given that most of the origami detected had more than five errors (FIG. 9E). We anticipate that reducing the number of errors by improving origami design and/or imager strand performance would allow more efficient algorithms for data recovery, which would in turn decrease the number of bits dedicated to error correction and thus increase information density.

Our fountain code algorithm is exceedingly robust to randomly lost packets of information, as long as the receiver receives K+£ packets, where K is the minimum number of packets required to encode the file under perfect conditions (i.e., K is equal to the file size) and is the number of additional packets received. The probability of being able to decode the file is then (1−δ), where δ is upper-bounded by 2\hat{ }(−Kε).²⁵ This equation implies that all things being equal, the larger the file size the greater the likelihood of successfully recovering the file at the receiver. Normally, the transmitter continues to transmit droplets in a fountain code until the receiver acknowledges successful file recovery. In the case of dNAM, this is not possible since the number of droplets must be fixed ahead of time to equal the number of origami. Reducing the error rates, or improving error correction/detection, would have the added benefit of reducing the number of droplets and hence origami discarded by the fountain code. These improvements would make it easier to determine the minimum number of droplets/origami needed to ensure robust file recovery while increasing information density even further.

The lower abundance and higher error rate of origami-2 (FIG. 9) indicates that some designs have defects that we could not detect by AFM or SRM alone. Careful defect analysis indicates that incorporated but inactive data domains play a greater role in producing errors than unincorporated staple strands²⁶. Future dNAM research should focus on sequence optimization to minimize variation in hybridization rates and the formation of off-target structures²⁷. It should also include the use of larger DNA origami and increased bit depth through multiplexing.

### Conclusion

DNA is an emerging material for data storage due to its high information density, high durability, low energy of operation, and the declining costs of synthesis¹. The traditional approach in the field is to design and synthesize unique oligos that encode data directly into their sequence. This data is recovered by reading the pool of oligos using sequencing. In contrast, dNAM takes advantage of another property of DNA—its programmability. By encoding binary data into DNA origami and reading it as spatially and temporally distinct hybridization events, dNAM decouples information recovery from sequencing. Editing the data is trivial through the inclusion or exclusion of sequence extensions from a library of staple strands. Data strands can be stored directly or incorporated into origami and then stored; separating the 3D storage density from the 2D reading density. In addition, dNAM is a massively parallel process because the large optical field of view affords tens of thousands of origami to be imaged simultaneously, and the number of optical read heads is proportional to the concentration of the imager strands. Rather than averaging thousands of DNA-PAINT images together, to resolve the digital data″, individual origami were read here using custom encoding, decoding, and error-correction algorithms. Our algorithms combined fountain codes with bi-level parity codes to significantly enhance our data retention—creating a multi-layer error correction scheme that encoded index, orientation, parity, and checksum bits into the origami. As a proof of concept, several bytes of data were recovered in a single DNA-PAINT recording. Even when the DNA origami recovery rate was poor (as low as 63%) the message was recovered 100% of the time. As a technology platform, dNAM offers a new pathway to harnessing the advantages of DNA as a material for information storage.

### Materials and Methods

The materials purchased for this study, and their respective vendors, are outlined below. All other reagents were obtained from Sigma.

| Materials Purchased | Vendor |
|---|---|
| DNA Staple Strands | Integrated DNA |
| | Technologies |
| M13 bacteriophage single-stranded | Bayou Biolabs |
| DNA scaffolds (M13mp18) | |
| Cy3B-labeled DNA oligonucleotide | Bio-Synthesis, Inc. |
| (M1 Imager strand: | |
| CTAGATGTAT-Cy3B) | |
| 150 nm diameter silanized gold | Nanopartz |
| nanoparticles (AuNPs) | |
| Glass coverslips | Ted Pella, Inc. |
| Sticky-Slide flow cells | Ibidi |
| (sticky-Slide I 0.2 Luer) | |
| Liquinox | Pollardwater, Inc. |
| MilliporeSigma | MilliporeSigma |
| Protocatechuate 3,4-dioxygenase | MilliporeSigma |
| pseudomonas (PCD) | |
| (+−)-6-hydroxy-2,5,7,8-tetra- | MilliporeSigma |
| methylchromane-2-carboxylic acid | |
| (Trolox) | |
| MgCl₂ | MilliporeSigma |
| Nuclease-free water | Thermo Fisher |
| | Scientific |
| Tris-borate-EDTA (TBE) | Thermo Fisher |
| | Scientific |
| Tris-Acetate-EDTA (TAE) | Thermo Fisher |
| | Scientific |

### Buffers

As previously described¹⁸, two buffers were used to prepare and image DNA origami: a deposition buffer and an imaging buffer. The deposition buffer contained 0.5×TBE and 18 mM MgCl₂. The imaging buffer contained the deposition buffer with the supplement of 60 nM PCD, 1 mM Trolox, 3 nM imager strands, and 10 mM PCA. PCA was added to the imaging buffer immediately before the start of a DNA-PAINT recording.

### Encoding Algorithm

The encoding algorithm used a multi-layer error correction scheme to encode message data bits along with index, orientation, and error correction bits onto multiple origami (FIG. 2).

At the message level, the algorithm used a fountain code to encode the data. Let m be a message string composed of a sequence of n bits. The fountain code algorithm first divides m into k equally sized and non-overlapping substrings s₁, s₂, . . . , sₖ, where the concatenation s₁s₂ . . . sₖ=m, and then systematically combines one to many segments using the binary XOR operation to form multiple data blocks called droplets. The number of segments d used to form each droplet are typically drawn from a distribution based on the Soliton distribution:

$$p ( 1 ) = 1 / k \\ p ( d ) = \frac{1}{d ( d - 1 )} for d = 2 , 3 , … , k . \qquad ( 1 )$$

The Soliton distribution ensures that the algorithm encodes the optimal number of single segment droplets necessary for the decode step. Once the number of segments d for a droplet is determined, the droplet is formed by XOR'ing d randomly selected, unique segments from m, with each segment being selected with probability 1/k.

For our experiments, we divided the message ‘Data is in our DNA!\n’ into 10 segments of 16 bits each. The segments were then combined via an XOR in different combinations using the fountain code algorithm to form the 15 droplets. While the theoretical minimum number of 16-bit droplets required to decode the message is 10, the redundancy provided by the additional droplets ensured that the message would be recoverable in all cases involving the loss of one droplet, and in some cases with the loss of up to five droplets (FIG. 10).

After generating the droplets using fountain codes, the encoding algorithm encoded each droplet onto 15 6×8 matrixes, and sequentially added index and orientation marker bits, computed and added checksum bits, and then added parity bits (FIG. 1B). These matrixes were used to construct 15 origami structures, with a one-to-one mapping between the matrixes and the origami.

FIG. 1A shows the layout of how droplet information was encoded onto each origami, composed of 16 bits of droplet data (green coloring in FIG. 1A), four indexing bits (red), four orientation bits (magenta), four checksum bits (yellow), and twenty parity bits (blue). It is important to note that the layout of the data, orientation, and index bits relative to the corresponding parity and checksum bits is invariant to rotation, which made it possible for the error correction algorithm to perform error detection and recovery before determining the orientation (FIGS. 2B-2C). This led to more robust data recovery.

### DNA Origami Folding

Rectangular DNA origami structures (˜90×70 nm) were designed based on previous work by Rafat et al.²⁸ with 48 potential docking strand sites arranged in a 6×8 matrix with 10 nm spacing. Then, using the protocol described by Schnitzbauer et al.¹⁸ a mixture of extended and unmodified staple strands were selected to fold the M13 scaffold into the designed shape, with extended strands located at the ‘1’ positions described in the design matrix (SI Table 51). As described in the introduction, an extended staple strand has a binding site for the M1 imager strand, unmodified strands bind solely to the scaffold DNA to induce folding. Using this method, 15 origami designs were created that matched the 15 matrixes output by the encoding algorithm.

We assembled individual origami designs by combining 22 nM M13mp18 with 10× unmodified stands, 50× extended strands, lx TAE and 18 mM MgCl₂ (in nuclease free water; 100 μL total volume) and folding in a Mastercycler nexus thermal cycler (Eppendorf) using the following heating cycle: [1 min 90° C., 2 min 80° C., then from 80° C. to 25° C. over 12 h]. We purified the origami by running them on an in ice-cooled 0.8% agarose gel containing 0.5×TBE and 8 mM MgCl₂, excising the single sharp band and collecting the exudate of the crushed gel piece. Sharp triangle origami used as fiducial markers were prepared similarly, as previously described²⁹. All purified origami was stored in the dark at 4° C. until use.

### Glass Coverslip Preparation

Borosilicate glass coverslips (25×75 and 22×22 mm, #1 Gold Seal Coverglass) were sonicated in 0.1% (v/v) Liquinox and nano-pure water (1 min in each) to remove contaminants and dried at 40° C. for at least 30 min. Fiducial markers (200 μL of 0.2 pM AuNPs) were deposited onto the coverslips for 10 min at room temperature. The labelled coverslips were rinsed with methanol and nano-pure water and stored at 40° C. prior to use.

DNA-Origami Deposition onto Coverslips

The glow discharge technique previously described by Green²⁶ was used to deposit DNA origami onto glass coverslips using an air-plasma vacuum glow-discharge system. Briefly, coverslips that had been cleaned and labelled with fiducial markers were exposed to glow discharge generated using an electrode coupled 115 V Electro-Technic BD-10A High Frequency Generator under 2 Torr of vacuum for 75 s. For DNA-PAINT analysis, a sticky-Slide flow cell (˜50 μL channel volume) was glued to the coverslip DNA origami deposited by introducing 200 μL of 0.05 nM origami (a mixture of dNAM origami, and sharp triangle origami²⁹ added as additional fiducial markers, in deposition buffer) into the flow chamber and incubated for 30 min at room temperature. After deposition, the flow chamber was rinsed with 1 mL of deposition buffer (no DNA origami) and refilled with imaging buffer.

When performing AFM measurements on samples previously used for DNA-PAINT, a custom fluid chamber, modified from Jungmann et al.30, was used. A 22×22 mm coverslip was glued to a microscope slide using double-sided sticky tape with the addition of a thin layer of gel sealant—to both seal any gaps and weaken the binding of tape to the glass. Once DNA-PAINT imaging had been performed the sealant allowed the coverslip to be easily removed for further AFM analysis.

### Fluorescence Microscopy

DNA origami were imaged below the diffraction-limit of light via DNA-PAINT18 using an inverted Nikon Eclipse Ti2 microscope from Nikon Instruments in total internal reflectance fluorescence (TIRF) mode. The images were acquired using an: integrated Perfect Focus System from Nikon Instruments; an oil-immersion CFI Apochromat 100×TIRF objective, with a 1.49 numerical aperture, plus an extra 1.5× magnification from Nikon Instruments; and a 405/488/561/647 nm Laser Quad Band Set TIRF filter cube from Chroma. A 561 nm laser source excited fluorescence from the DNA-PAINT imager strands within an evanescent field extending a few hundred nanometers above the surface of the glass coverslip. The emitted fluorescence was imaged onto the full chip with 512×512 pixels (1 pixel=16 μm) using a ProEM EMCCD camera from Princeton Instruments at a 300 ms exposure time (˜3 frames/s). During an experimental recording, each of the individual data strands, within a dNAM origami's matrix, transiently and repeatedly bound an imager strand, to emit a signal, creating a series of blinks. Images with blinking events were recorded into a stack (typically 40,000 frames per recording) using Nikon NIS-Elements version 5.20.00 from Nikon Instruments prior to processing and analysis.

### DNA-PAINT Fluorophore Localization

After recording a DNA-PAINT stack, the center position of signals (a.k.a localizations) emitted by imager probes, transiently binding to DNA-origami docking strands, were identified using the ImageJ ThunderSTORM plugin³¹. The localizations were rendered and then drift corrected using the Picasso-Render software package, as described by Schnitzbauer et al.¹⁸. Data visualization and peak fitting of image data for PSF analysis were performed using OriginPro Version 2019b³².

### Localization Data Processing

A custom algorithm was developed for identifying clusters of localizations, determining the maximum likelihood position of the emitters, and generating binary matrix data. The algorithm selected localization clusters at random from the localization list. To do this, it sampled random points in the scene, found the average position of nearby localizations, and counted the localizations within a radius (R) and the localizations within a band R<r<2R. The algorithm accepted clusters if the counts in the inner circle were greater than a threshold and the counts in the outer band were less than 15% of the counts in the inner band. This ensured selection of bright clusters that were isolated from other clusters.

The algorithm then fit the cluster localizations to a grid of emitters. An idealized grid was created using the average DNA-PAINT image produced by several thousand individual origami structures of the same architecture used in this work. The algorithm performed fitting using a maximum likelihood estimation for the likelihood function:

$$L ( I , {x}_{c} , {y}_{c} , θ , Δ {x}_{g}^{2} , B ) = {\prod }_{i} ( {\sum }_{k} \frac{{I}_{k}}{a} exp ( - \frac{{( {x}_{i} - {x}_{k} ( {x}_{c} , {y}_{c} , θ ) )}^{2} + {( {y}_{i} - {y}_{k} ( {x}_{c} , {y}_{c} , θ ) )}^{2}}{Δ {x}_{i}^{2} + Δ {x}_{g}^{2}} ) ) * \frac{B}{A} * P ( N , I , B ) \qquad ( 2 )$$

Where I^{k} is the intensity of the k^{th} emitter, (x_{c}, y¬_{c}) is the center position of the grid, θ is the rotation angle of the grid, Δx_{g} is the global lateral uncertainty caused by error in drift correction, B is the background, Δxᵢ is the lateral position uncertainty of localization i reported by the ThunderSTORM analysis described above, (xᵢ, yᵢ) is the position of the i^{th} localization, (xₖ,yₖ) is the position of the k^{th} emitter, as a function of the center position and rotation of the grid, A is the area of the cluster, and N is the number of localizations found in the cluster. a is a normalization constant given by:

α=2π(Δxᵢ²+Δx_{g}²) (3)

P(N,I,B) is the probability of finding N localizations given the intensity of each grid point and the background intensity, determined from the Poisson distribution of mean value N. This likelihood function determines the probability of finding localizations at all of the observed sites given a set of point emitters at the grid sites with intensity I^{k} and background intensity B. The optimization utilized the L-BFGS-B method of the minimize function provided by Scipy³³ to minimize -log(L) subject to the constraint that all intensities are positive. Signals that did not align to the 6×8 grid were filtered to minimize fragmented origami and to reduce inadvertent assimilation of the triangular origami fiducial markers into the results.

The algorithm then assigned the emitters a binary value (1 or 0) using an empirically derived threshold value. This binary matrix data was decoded using the decoding algorithm described below.

In parallel with this blind cluster analysis, the processing algorithm also carried out a template matching step to more reliably identify individual origami and analyze their errors. This additional step used the known origami designs as templates, matching the observed origami to the best fit, based on the total number of errors. This method was more robust to higher error rates than the blind cluster analysis and allowed more origami to be identified for image averaging and error analysis (see FIGS. 9D-9E). It should be noted, however, that the template matching method cannot be considered as a data reading method because it requires a priori knowledge of the data being analyzed. For this reason, none of the analysis of the recovery rates or data density discussed here used data obtained from pattern matching.

### Decoding Algorithm

The decoding algorithm (FIG. 6) utilized a multi-layer error correction/encoding scheme to recover the data in the presence of errors. The algorithm first works at the dNAM origami level (Step 1, below), using the parity and checksum bits, to attempt to identify and correct errors and recover the correct matrix. After recovery, the algorithm uses binary operations to recover the original data segments from the droplets (Step 2, below).

### Decoding Algorithm: Step 1—Error Correction

Given raw binary matrix data M for a single dNAM origami, output from the localization data processing step, the matrix decoding algorithm determined which, if any, bits were associated with checksum and parity errors by calculating the bi-level matrix parity and checksum values, as described in FIGS. 2B-2C. Any discrepancies between the calculated parity and checksum values and the values recovered from the origami were noted, and a weight for each of the bits associated with the errant parity/checksum calculation was deduced. If no parity/checksum errors were detected for a particular matrix, then the data was assumed to be accurate, and the algorithm proceeded to extract the message data.

To determine the site(s) of likely errors, the decoding algorithm first determined a weight for every cell in M, beginning with data cells (the cells containing droplet, index, or orientation bits) and proceeding to parity and checksum cells. Let P_{c}_{ᵢⱼ} be the set of parity functions calculated over a given data cell cᵢⱼ. Then for each data cell cᵢⱼ:

$${x}_{i j} = {Σ}_{{f}_{{c}_{p q}} ∈ {P}_{{c}_{ij}}} \vert {c}_{p q} - {f}_{{c}_{p q}} ( M ) \vert \qquad ( 4 )$$

Where c_{pq} is the parity cell where the expected binary value off is stored.

The weight for each parity cell cᵢⱼ was then calculated based on the number of non-zero weights greater than 1 for the data cells associated with it. More formally, let cᵢⱼ be a parity cell and D_{c}_{ᵢⱼ} be the set of data cells used in the calculation of cᵢⱼ. Then the weight xᵢⱼ for each parity cell cᵢⱼ is:

$${x}_{i j} = {\sum }_{{c}_{p q} ∈ {D}_{{c}_{i j} ⩓ {x}_{p q} > 1}} s g n ( {x}_{p q} ) \qquad ( 5 )$$

The higher the weight value, the higher the probability that the corresponding cell had an error. An overall score for the matrix was then calculated by summing over all x_{i,j} and normalizing by the sum of the correctly matched parity bits. This value was designated as the overall weight of the matrix. Higher values of this weight correspond to matrixes with more errors.

$$Overall matrix weight = \frac{{\sum }_{i = 0}^{6} {\sum }_{j = 0}^{8} {x}_{i j}}{\# number of matched parity bits} \qquad ( 6 )$$

The algorithm then performed a greedy search to correct the errors using a priority queue ordered by the overall matrix weight (FIG. 7). The algorithm began by iteratively altering each of the probable site errors and computing the overall matrix weight of the modified matrix for each, placing each potential bit flip into a priority queue where the flips that produced the lowest overall weights had the highest priority. At each step, the algorithm selected the bit flip associated with the highest priority in the queue and then repeated this process on the resulting matrix. This process was continued until the algorithm produced a matrix with no mismatches or until it reached the maximum number of allowed bit flips (9 for our simulation/experiment). If it reached the maximum number of flips, it returned to the queue to pursue the next highest priority path. If the algorithm found a matrix with no mismatches, it then checked the orientation bits and oriented the matrix accordingly. The droplet and index data were then extracted and passed to the next step. If the queue was emptied without finding a correct matrix, the algorithm terminated in failure.

### Decoding Algorithm: Step 2—Fountain Code Decoding

After extracting the droplet and index data from multiple matrixes the algorithm attempted to recover the full message (FIG. 12). Once decoded, each droplet had one or multiple segments XORed in it. Using the recovered indexes, the algorithm determined how many and which segments were contained in each droplet. To decode the message, the algorithm maintained a priority queue of droplets based on the number of segments they contained (their degree), with the lowest degree droplets having the highest priority. The algorithm looped through the queue, removing the lowest degree droplet, attempting to use it to reduce the degree of the remaining droplets using XOR operations, and re-queuing the resulting droplets. Upon finding a droplet of ‘degree one’ it stored it as a segment for the final message. If all segments were recovered, the algorithm terminated successfully.

### Data Simulation Test

To test the robustness of our encoding and decoding algorithms, origami data were simulated with randomly generated messages and errors. First, random binary messages of size m were created (for m=160 to 12,800 bits, at 320-bit intervals). These messages were then divided into m/b equally sized segments, where b is the number of data bits to be encoded onto an individual origami. For fixed-size origami, larger messages necessitated a smaller b, as more bits had to be dedicated to the index. In these cases, b varied between eight (for m=12,800) and twelve (for m=160). After determining message segments, droplets were formed using the fountain code algorithm and encoded onto origami, along with the corresponding index, orientation, and error-correcting bits. Ten in silico copies of each unique origami were created, and 0-9 bits flipped at random to introduce errors. The origami were decoded as described above.

### Code Availability

DNA-PAINT images were analyzed using custom and publicly available codes (as indicated). The encoding/decoding algorithms were written in-house using Python, version 3.7.334. The source codes for the encoding, decoding and localization algorithms are available on GitHub at https://github.com/gmortuza/dnam.

The schematics in FIGS. 1A-1B of digital Nucleic Acid Memory were derived from a model created using Nanodesign (www.autodeskresearch.com/projects/nanodesign).

### AUTHOR CONTRIBUTIONS

W. L. H. conceived the concept. E. J. H., T. A., W. K., E. G., R. Z., and W. L. H. designed the study. C. W., E. J. H., T. A., W. K., E. G., and W. L. H. supervised the work. C. W. managed the research project. G. D. D. and L. synthesized the DNA origami and performed DNA-PAINT imaging. L. P. carried out AFM imaging and analysis. T. A. and G. M. M developed the encoding-decoding algorithms and necessary software, performed data processing, and generated the simulations. G. D. D. and W. C. developed the image-analysis software and analyzed the DNA-PAINT recordings. C. M. G. performed preliminary experiments and contributed critical suggestions to experimental design. All authors prepared the manuscript.

### REFERENCES

1. Victor Zhirnov. 2018 Semiconductor Synthetic Biology Roadmap. 36 (2018) doi:10.13140/RG.2.2.34352.40960.
2. ITRS. International Technology Roadmap for Semiconductors, 2015 Results. Itrpv 0, 1-37 (2016).
3. Reinsel, D., Gantz, J. & Rydning, J. The Digitization of the World—From Edge to Core. IDC White Pap. U.S. Pat. No. 44,413,318 (2018).
4. Zhirnov, V., Zadegan, R. M., Sandhu, G. S., Church, G. M. & Hughes, W. L. Nucleic acid memory. Nat. Mater. 15, 366-370 (2016).
5. Carlson, R. Time for New DNA Sequencing And Synthesis Cost Curves. 1-31 https://synbiobeta.com/time-new-dna-synthesis-sequencing-cost-curves-rob-carlson/(2014).
6. Organick, L. et al. Random access in large-scale DNA data storage. Nat. Biotechnol. 36, 242-248 (2018).
7. Goldman, N. et al. Towards practical, high-capacity, low-maintenance information storage in synthesized DNA. Nature 494, 77-80 (2013).
8. Grass, R. N., Heckel, R., Puddu, M., Paunescu, D. & Stark, W. J. Robust chemical preservation of digital information on DNA in silica with error-correcting codes. Angew. Chemie—Int. Ed. 54, 2552-2555 (2015).
9. Bornholt, J. et al. A DNA-Based Archival Storage System. ACM SIGARCH Comput. Archit. News 44, 637-649 (2016).
10. Shipman, S. L., Nivala, J., Macklis, J. D. & Church, G. M. Molecular recordings by directed CRISPR spacer acquisition. Science (80). 353, (2016).
11. Erlich, Y. & Zielinski, D. DNA Fountain enables a robust and efficient storage architecture. Science (80). 355, 950-954 (2017).
12. Blawat, M. et al. Forward error correction for DNA data storage. Procedia Comput. Sci. 80, 1011-1022 (2016).
13. Yazdi, S. M. H. T., Gabrys, R. & Milenkovic, O. Portable and Error-Free DNA-Based Data Storage. Sci. Rep. 7, 1-6 (2017).
14. Lee, H., Kalhor, R., Goela, N., Bolot, J. & Church, G. Enzymatic DNA synthesis for digital information storage. bioRxiv 348987 (2018) doi:10.1101/348987.
15. Wang, P., Meyer, T. A., Pan, V., Dutta, P. K. & Ke, Y. The Beauty and Utility of DNA Origami. Chem 2, 359-382 (2017).
16. Nieves, D. J., Gaus, K. & Baker, M. A. B. DNA-based super-resolution microscopy: DNA-PAINT. Genes (Basel). 9, 1-14 (2018).
17. Jungmann, R. et al. Single-molecule kinetics and super-resolution microscopy by fluorescence imaging of transient binding on DNA origami. Nano Lett. 10, 4756-4761 (2010).
18. Schnitzbauer, J., Strauss, M. T., Schlichthaerle, T., Schueder, F. & Jungmann, R. Super-resolution microscopy with DNA-PAINT. Nat. Protoc. 12, 1198-1228 (2017).
19. Luby, M. LT Codes. in Proceedings of the 43rd Annual IEEE Symposium on Foundations of Computer Science (IEEE, 2002) 271-280 (2002).
20. MacKay, D. J. C. Fountain codes. IEE Proc.—Commun. 152, 1062-1068 (2005).
21. Greengard, S. The future of data storage. Commun. ACM 62, 12-12 (2019).
22. Gwosch, K. C. et al. MINFLUX nanoscopy delivers 3D multicolor nanometer resolution in cells. Nat. Methods 17, (2020).
23. Wade, 0. K. et al. 124-Color Super-resolution Imaging by Engineering DNA-PAINT Blinking Kinetics. Nano Lett. 19, 2641-2646 (2019).
24. Takabayashi, S. et al. Boron-implanted silicon substrates for physical adsorption of DNA origami. Int. J. Mol. Sci. 19, (2018).
25. Langari, S. M. M., Yousefi, S. & Jabbehdari, S. Fountain-code aided file transfer in vehicular delay tolerant networks. Adv. Electr. Comput. Eng. 13, 117-124 (2013).
26. Green, C. Nanoscale Optical and Correlative Microscopies for Quantitative Characterization of DNA Nanostructures. Journal of Chemical Information and Modeling vol. 53 (Boise State University, 2019).
27. Hata, H., Kitajima, T. & Suyama, A. Influence of thermodynamically unfavorable secondary structures on DNA hybridization kinetics. Nucleic Acids Res. 46, 782-791 (2018).
28. Aghebat Rafat, A., Pirzer, T., Scheible, M. B., Kostina, A. & Simmel, F. C. Surface-assisted large-scale ordering of DNA origami tiles. Angew. Chemie—Int. Ed. 53, 7665-7668 (2014).
29. Rothemund, P. W. K. Folding DNA to create nanoscale shapes and patterns. Nature 440, 297-302 (2006).
30. Dai, M., Jungmann, R. & Yin, P. Optical imaging of individual biomolecules in densely packed clusters. Nat. Nanotechnol. 11, 798-807 (2016).
31. Ovesný, M., Kř\check{i}žek, P., Borkovec, J., Svindrych, Z. & Hagen, G. M. ThunderSTORM: A comprehensive ImageJ plug-in for PALM and STORM data analysis and super-resolution imaging. Bioinformatics 30, 2389-2390 (2014).
32. OriginLab Corporation. OriginPro Version 2019b.
33. Oliphant, T. E. Python for scientific computing. Comput. Sci. Eng. 9, 10-20 (2007).
34. Python Software Foundation. Python Language Reference, version 3.7.3. http://www.python.org.

### Supplemental Materials and Methods

### Encoding/Decoding Algorithms

See attached diagrams and flowcharts for graphical representation of the main steps of the algorithms. Table S1 lists the different designs generated by the encoding algorithm for the message ‘Data is in our DNA!\n’.

TABLE S1
Origami Designs

| | Binary | | Matrix | | Binary | | Matrix | | Binary | | Matrix |
|---|---|---|---|---|---|---|---|---|---|---|---|
| Index | Index | Droplet | Design | Index | Index | Droplet | Design | Index | Index | Droplet | Design |
| 0 | 0000 | 00110110 | 00110110 | 5 | 0101 | 01011111 | 01011111 | 10 | 1010 | 01101110 | 01101110 |
| | | 01010101 | 11110111 | | | 01001011 | 11010101 | | | 00000101 | 11010101 |
| | | | 00111010 | | | | 10111000 | | | | 00000110 |
| | | | 00110111 | | | | 00101001 | | | | 11011110 |
| | | | 11011110 | | | | 11000010 | | | | 11111100 |
| | | | 00101010 | | | | 10110100 | | | | 01101000 |
| 1 | 0001 | 00100110 | 00100110 | 6 | 0110 | 00010000 | 00010000 | 11 | 1011 | 00010001 | 00010001 |
| | | 01100000 | 10101001 | | | 01101001 | 11101011 | | | 00001010 | 11100111 |
| | | | 10110100 | | | | 00011010 | | | | 10011010 |
| | | | 00011101 | | | | 11100011 | | | | 10000000 |
| | | | 11111100 | | | | 10000110 | | | | 10101110 |
| | | | 00000001 | | | | 10100101 | | | | 01010100 |
| 2 | 0010 | 01011111 | 01011111 | 7 | 0111 | 01010100 | 01010100 | 12 | 1100 | 01001110 | 01001110 |
| | | 00111010 | 11011001 | | | 00001000 | 10100001 | | | 01000110 | 11001101 |
| | | | 00011100 | | | | 11100000 | | | | 01000100 |
| | | | 10111100 | | | | 11011000 | | | | 00000001 |
| | | | 11100100 | | | | 11011000 | | | | 11000100 |
| | | | 00010111 | | | | 10000100 | | | | 11011000 |
| 3 | 0011 | 00100001 | 00100001 | 8 | 1000 | 00100000 | 00100000 | 13 | 1101 | 01010010 | 01010010 |
| | | 01010110 | 10000101 | | | 01101001 | 10111111 | | | 01110110 | 10011111 |
| | | | 10011110 | | | | 01000100 | | | | 11001100 |
| | | | 10010001 | | | | 01010011 | | | | 01010011 |
| | | | 11100010 | | | | 11100100 | | | | 11001110 |
| | | | 00011010 | | | | 01100101 | | | | 11011011 |
| 4 | 0100 | 00011010 | 00011010 | 9 | 1001 | 00100000 | 00100000 | 14 | 1110 | 00001010 | 00001010 |
| | | 00010010 | 11111011 | | | 01101111 | 11011011 | | | 01111101 | 11010101 |
| | | | 00111110 | | | | 10101100 | | | | 01101100 |
| | | | 00010000 | | | | 00010011 | | | | 10001011 |
| | | | 11011000 | | | | 11010000 | | | | 10010100 |
| | | | 10010010 | | | | 01111101 | | | | 11101111 |

The binary data droplets and data strings associated with each origami index are shown.

Atomic Force Microscopy

AFM analysis was conducted on freshly cut mica substrates or glass coverslips (prepared as described above). 4 μL of a dNAM origami sample was deposited onto the substrate for 5 min and then 100 μL of deposition buffer added to form a droplet on top of the sample. AFM imaging was performed with a Dimension-FastScan system from Bruker set to amplitude modulation mode. Imaging was carried out in liquid with a set-point ratio between the free amplitude and imaging amplitude of ˜0.7. The FastScan D cantilever was supplied by Bruker, with a nominal spring constant of 0.25 N/m. Sub-nanometer amplitude was used to image DNA docking strand positions on every origami structure following the method of l. Tilt correction (line or plane flattening) was performed using WSxM software package² (Nanotec Electronica, Madrid, Spain) and a low-pass filter applied to remove noise. Further filtering, using inverse FFT band rejection, was added to visually highlight the docking strands.

### Supplemental Results

### DNA-PAINT Resolution

To evaluate the resolution of the DNA-PAINT experiments, FWHM values were derived by taking transect measurements centered on binding sites in rendered images (with 1-pixel blur applied) of either individual or ‘averaged’ dNAM origami (FIGS. 4A-4D). In both cases at least ten binding sites were examined for each structure using with horizontally or vertically aligned positioned transects (FIGS. 4A-4B). FWHM values of 6.6 nm±1.6 SD (single origami images, n=124) and 7.2 nm±0.3 SD (averaged origami images, n=47) were calculated from Gaussian fits to plots of the transect data (FIGS. 4C-4D).

### Proximity Error Analysis

Analysis of our error locations (FIGS. 8A-8C) showed slightly higher false negative error rates around the edges of dNAM origami, but there was no pattern of error locations in the origami that would explain the variance in error rates between different origami designs. There is a correlation between a higher number of 1-bits and a higher number of false negatives, as would be expected, but this does not explain most of the observed variance between origami. The phenomenon of higher errors near the edges of the origami has been observed previously³ and was interpreted as reflecting a difference in staple strand incorporation efficiencies. To investigate this and other sources of potential sources of error in our array designs, we performed atomic force microscopy (AFM) imaging on individual origami deposited on mica (FIG. 5). From the averaged SRM images in FIG. 3, it can be seen that every data strand was recorded at least once for all expected positions in all arrays. This suggests that there were no systematic failures in strand incorporation or data strand binding domains. This is further substantiated by the AFM images, in which origami were typically both well formed (lacking holes and having the expected dimensions) and appeared to have incorporated the majority of their data strands. Although it was possible to resolve the majority of data strands positions (FIG. 5), a strict analysis on missing data strands using AFM would not be completely reliable since tip-sample interactions could easily promote strand compression and displacement. However, our previous correlative defect analysis of DNA origami, combining AFM and DNA-PAINT, indicated that strand incorporation plays a role in origami site yields and defects are likely due to the unavailability of incorporated staple strands. Further, DNA-PAINT itself may locally increase the susceptibility of DNA origami to damage during imaging⁴. This is in keeping with our results and suggests that further optimization of the DNA-PAINT imaging protocol will help reduce the false negative error rate.

### SUPPLEMENTAL REFERENCES

1. Miller, E. J. et al. Sub-nanometer resolution imaging with amplitude-modulation atomic force microscopy in liquid. J. Vis. Exp. 2016, 1-10 (2016).
2. Horcas, I. et al. WSXM: A software for scanning probe microscopy and a tool for nanotechnology. Rev. Sci. Instrum. 78, (2007).
3. Strauss, M. T., Schueder, F., Haas, D., Nickels, P. C. & Jungmann, R. Quantifying absolute addressability in DNA origami with molecular resolution. Nat. Commun. 9, 1-7 (2018).
4. Green, C. Nanoscale Optical and Correlative Microscopies for Quantitative Characterization of DNA Nanostructures. Journal of Chemical Information and Modeling vol. 53 (Boise State University, 2019).

What is claimed is:

1. A nucleic acid memory architecture for data storage, comprising:
a population of nucleic acid strands, wherein the population of nucleic acid strands comprises of data strands having an optional docking domain located at a data sites and structural strands and wherein the population of nucleic acid strands forms an indexed array when self-assembled.

2. The nucleic acid memory architecture of claim 1, wherein the architecture is an origami, molecular canvas, single stranded tiles, or single stranded oligomer.

3. The nucleic acid memory architecture of claim 2, wherein the architecture is an origami.

4. The nucleic acid memory architecture of claim 1, further comprising an image strand, wherein a dye is bound to the image strand.

5. The nucleic acid memory architecture of claim 4, wherein the image strand is bound to the optional docking domain.

6. The nucleic acid memory architecture of claim 5, wherein the data site is a data bit, an index bit, an orientation bit, a parity bit, a checksum bit, and/or combinations thereof.

7. The nucleic acid memory architecture of claim 5, wherein the data site is represented by a (1) when the image strand is bound to the data strand.

8. The nucleic acid memory architecture of claim 5, wherein the data site is represented by a (0) when the data strand lacks the optional docking domain.

9. The nucleic acid memory architecture of claim 2, wherein the origami is a rectangular 2D nanostructure.

10. The nucleic acid memory architecture of claim 1, wherein the nucleic acid is DNA, RNA, BNA, UNA, LNA, and/or combinations thereof.

11. The nucleic acid memory architecture of claim 10, wherein the nucleic acid is DNA.

12. The nucleic acid memory architecture of claim 5 wherein the docking domain between a data strand and an image strand is partially complemented.

13. The nucleic acid memory architecture of claim 4, wherein the image strand is multiplexed.

14. A method of encoding data onto a nucleic acid architecture, comprising:
encoding a data set into a binary string; and
assigning the binary string to a plurality of indexed positions on a nucleic acid architecture, wherein the nucleic acid architecture forms an indexed array.

15. The method of claim 14, wherein the encoding is a rateless code.

16. The method of claim 15, wherein a population of segments is calculated using Equation (1).

17. The method of claim 16, wherein the segments of a data block are combined using XOR.

18. The method of claim 14, further comprising assigned index bits and/or orientation bits to the nucleic acid architecture.

19. The method of claim 18, further comprising:
calculating an error bit; and
assigning the error bit to the nucleic acid architecture.

20. The method of claim 18, wherein the error bit is a checksum and/or parity bit.

21. A method of recovering data from a nucleic acid architecture, comprising:
capturing an image of the nucleic acid architecture, wherein the nucleic acid architecture is an indexed array and bound to an image strand comprising an excited chromophore;
processing the image; and
extracting the data.

22. The method of claim 21, wherein capturing is through microscopy.

23. The method of claim 21, further comprising correcting errors, wherein the nucleic acid architecture comprises an error bit.

24. The method of claim 23, wherein the error bit is a checksum bit, parity bit, and/or orientation bit.

25. The method of claim 24, wherein correcting errors is a bi-level, parity based, and orientation-invariant error detection scheme.

26. The method of claim 25, wherein the bi-level, parity based, and orientation-invariant error detection scheme is calculated with any one of Equations (4), (5) or (6).

27. The method of claim 21, further comprising extracting index bit, droplet bits, and/or combinations thereof.

28. The method of claim 21, wherein the data is encoded by a fountain code, further comprising:
extracting segment information; and
decoding the fountain code.

29. The method of claim 22, wherein the capturing of an image is done by super resolution microscopy.

30. The method of claim 29, wherein the super resolution microscopy is DNA-PAINT.

31. A method of stable data storage, comprising:
encoding a data set into a binary string;
assigning the binary string to an indexed array comprising a nucleic acid architecture, comprising of data strands and structural strands; and
storing the nucleic acid architecture.

32. The method of claim 31, further comprising encapsulating the data strands and/or the structural strands of the nucleic acid architecture.

33. The method of claim 32, wherein the data strands and/or the structural strands nucleic acid architecture is encapsulated in a silicate nanoparticle.

34. The method of claim 32, wherein storing is at ambient temperatures.

35. The method of claim 31, wherein storing is in a freezer.

36. The method of claim 31, wherein storing is in liquid nitrogen.

37. The method of claim 31, wherein the nucleic acid architecture is single strand tiles, origami, or molecular brick canvases.

38. The method of claim 31, wherein the nucleic acid architecture further comprises error bits.

39. A system for encoding data onto a nucleic acid architecture, comprising:
a device configured to synthesis the nucleic acid architecture;
a computer processor programmed to (i) encode the data into a binary sting, (ii) select data strands comprising docking domains, and (iii) construct an indexed array of the binary string.

40. The system of claim 39, wherein the processor is programmed to further perform (i) creating a rateless code, (ii) creating index bits and translate into the binary string, (iii) creating orientation bits and translate into the binary string, (iv) calculating parity bits and translate into the binary string, (v) calculating checksum bits and translate into the binary string, and combinations thereof.

41. A system for reading data from an indexed array, comprising:
a microscope configured to detect the presence or absence of a chromophore at an indexed position; and
a computer processors programmed to (i) capture the image from the microscope, (ii) identify the status of the indexed, (iii) generate a plurality of symbols from the data strands in (ii), and (iv) compile the information from the plurality of symbols.
